(19) United States
(12) Patent Application Publication
(10) Pub. No.: US 2025/0152912 A1
(43) Pub. Date: May 15, 2025

(54) PACKAGED CATHETER ASSEMBLY AND METHOD OF MANUFACTURING SAME

(71) Applicant: ConvaTec Limited, Flintshire (GB)

(72) Inventors: Patrik Lacko, Michalovce (SK); Branislav Meciar, Michalovce (SK); Vladimir Jenco, Michalovce (SK); Peter Fenovcik, Michalovce (SK)

(21) Appl. No.: 19/020,039

(22) Filed: Jan. 14, 2025

Related U.S. Application Data
(63) Continuation of application No. PCT/GB2023/051823, filed on Jul. 12, 2023.
(60) Provisional application No. 63/391,182, filed on Jul. 21, 2022.

(30) Foreign Application Priority Data
Aug. 8, 2022 (GB) GB2211512.5

(51) Int. Cl. A61M 25/00

(52) U.S. Cl. CPC A61M 25/0009; A61M 25/0017; A61M 25/002; A61M 25/0041; A61M 2205/0216; A61M 2205/025; A61M 2207/10; A61M 2209/06

(57) ABSTRACT

A catheter assembly that has a catheter tube coupled to a funnel, and a handling sleeve. The handling sleeve is formed from a flexible film material and is releasably attached to the funnel. One embodiment of the invention provides a method of processing a catheter assembly; the method comprising: engaging the funnel of a catheter assembly comprising a catheter tube and a funnel, such that the catheter tube hangs down from the funnel; and releasably attaching the handling sleeve to the funnel.

### TECHNICAL FIELD OF THE INVENTION

The present invention relates to packaged catheter assemblies. In particular to packaged urinary catheter assemblies, and most particularly to packaged intermittent male urinary catheter assemblies. It also concerns a manufacturing method.

### BACKGROUND TO THE INVENTION

Many people suffer from urinary problems that can make it difficult to pass urine. Intermittent catheter assemblies can provide a convenient and portable solution to this problem as a user is able to self-catheterise to relieve themselves as required. This reduces the effect of their urinary condition on their life and allows thm to enjoy a relatively normal lifestyle.

Intermittent catheters are typically single-use devices that are carried by the user then unpackaged and used as required.

To reduce the risk of damage to the urinary tract and improve comfort while using the catheter, it is also important they are lubricated before use. However, once lubricated and used a catheter can be slippery and difficult to handle.

Existing intermittent catheters such as the Coloplast SpeediCath Flex (RTM) and the Hollister VaPro (RTM) catheters disclose continuous film handling sleeves that cover the entire length of the catheter. While these provide some assistance to handling, the sleeve must be bunched up against one end of the catheter during use which can be difficult and unhygienic as dirt may get onto the catheter prior to use during bunching of the sleeve.

As such, full-length sleeves may not in fact be ideal.

Short sleeves have been proposed in the patent literature, with US2015/0352321 (HOLLISTER) proposing an embodiment (in FIGS. 26A and 26B thereof, which has a protective introducer tip arranged at the proximal end of the catheter with a short sleeve attached thereto. The short sleeve has a small, fixed portion, apparently permanently attached to the introducer tip and a free portion, detachable from the fixed portion by means of perforations. As such, the free portion can be detached from the fixed portion and the user can handle the catheter by holding the introducer tip in one hand and the free portion of the sleeve in the other hand.

Applying a full-length sleeve to a catheter before packaging it can be a fiddly job, it can involve a worker manually applying the sleeve to the catheter, then coiling the sleeved catheter and introducing it to a package. This is repetitive, low-skill, work and the use of manual labour introduces the possibility of contamination.

No indication is provided in US2015/0352321 as to how to produce the introducer tip with its detachably attached short sleeve, or how to apply it to a catheter. However, one can imagine that the introducer tip would be formed, then the short sleeve attached thereto, followed by attachment of the introducer tip, with the attached short sleeve to the end of the catheter tube. The introducer tip clips into the eyelets in the catheter tube, so this seems the most likely approach, and would involve opening the sleeve and threading the catheter tube through it. Notably, the sleeve is formed of two film sheets attached at their edges, so likely, it seems to be urged towards a flat configuration, with the sheets lying against each other. As such, manufacture of this embodiment appears liable to be complicated and time consuming. This may explain why no such product has reached the market.

It is an object of embodiments of the present invention to at least partially overcome or alleviate the above problems.

### SUMMARY OF THE INVENTION

According to a first aspect of the invention there is provided a method of processing a catheter assembly comprising a catheter tube coupled to a funnel and a handling sleeve releasably attached to the funnel; the method comprising engaging the funnel, such that the catheter tube hangs down from the funnel.

The method preferably comprises engaging the funnel, such that the catheter tube hangs down from the funnel and releasably attaching the handling sleeve thereto.

As such, one embodiment of the invention provides a method of processing a catheter assembly; the method comprising: engaging the funnel of a catheter assembly comprising a catheter tube and a funnel, such that the catheter tube hangs down from the funnel; and releasably attaching the handling sleeve to the funnel.

Advantageously, with a handling sleeve attached to the funnel of a catheter assembly, and the catheter assembly arranged such that the catheter tube hangs down from the funnel, the catheter assembly can be processed in a more automated fashion. Attachment of the handling sleeve to the funnel means that the handling sleeve will not fall off if the catheter assembly is hung from its funnel, which in turn allows processing to occur whilst hanging the catheter assembly vertically. This in turn allows for better use of space in a manufacturing facility, better alignment of catheter assemblies and so forth.

The handling sleeve may be attached via a sleeve-attachment portion.

As such, one embodiment of the invention provides a method of processing a catheter assembly comprising a catheter tube coupled to a funnel and a handling sleeve releasably attached to the funnel via a sleeve attachment portion; the method comprising engaging the funnel, such that the catheter tube hangs down from the funnel.

The handling sleeve (and optionally the sleeve-attachment portion) may be formed of a flexible film. A point of weakness may be provided between the handling sleeve and the sleeve-attachment portion. For example, the handling sleeve (and optionally the sleeve-attachment portion) may be formed of a plastics film. The handling sleeve may be made from any one or more of: polypropylene (PP), low density polyethylene (LDPE), high density polyethylene (HDPE) or biaxially orientated polypropylene (BOPP), for example. The catheter and/or pouch may be made from the same or different materials as the handling sleeve(s) and/or handling sleeve elements. In a preferred embodiments, the catheter is a hydrophilic thermoplastic elastomer (TPE), and the handling sleeve(s) and/or handling sleeve elements are LDPE. The funnel may be polyvinyl chloride (PVC). Thus, the handling sleeve and well as the catheter and funnel can be made from suitable materials.

The handling sleeve is preferably a short sleeve. A short sleeve as defined herein is a sleeve that (when attached) does not extend all the way to the tip of the catheter tube. For example, when attached, the handling sleeve may extend along less than 70%, less than 60%, or less than 50% of the distance to the tip of the catheter tube. A distance of at least 30%, 40% or 50% from the tip of the catheter towards the funnel may be exposed.

As such, one embodiment of the invention provides a method of processing a catheter assembly comprising a catheter tube coupled to a funnel and a handling sleeve releasably attached to the funnel, wherein the handling sleeve is a short sleeve which extends extend along less than 50% of the distance to the tip of the catheter tube, whereby a distance of at least 50% from the tip of the catheter towards the funnel is exposed; the method comprising engaging the funnel, such that the catheter tube hangs down from the funnel.

The method may comprise engaging the funnel with processing rails. In one embodiment the catheter assembly hangs from the processing rails, suspended by the funnel. Alternatively, the method may comprise suspending the funnel from suspension means, wherein the suspension means are engaged with the processing rails. For example, the suspension means may be a cradle, or a gripping means, such as pair of gripping arms.

The method may comprise picking a catheter from a plurality of catheters in bulk. The method may comprise separating and orienting a plurality of catheters in bulk, then picking a catheter. The separation and orientation may comprise orienting the catheter assemblies vertically (i.e. such that their axis is substantially vertical). For example, the separation and orientation may comprise suspending the catheters, by their funnels from a pair of infecd rails.

The method may comprise transitioning the catheters from the infeed rails to the processing rails. In one embodiment, the suspension means may pick a catheter from the infeed rails to transition it to the processing rails.

The method may comprise advancing the catheter assembly along the rails. For example, the method may comprise advancing the catheter assembly along the rails by vibrating the rails. The rails may be arranged at an angle to assist in the advancing of the catheter assembly by vibrating the rails.

The method may comprise advancing the catheter assembly along the rails to a sheathing station. The method may comprise attaching the handling sleeve to the funnel in the sheathing station. The method may comprise forming the handling sleeve in the sheathing station. For example, the method may comprise wrapping a sheet of film material around the catheter tube and joining opposing elongate edges of the sheet to form the handling sleeve. In another example, the method may comprise sandwiching the catheter tube between two sheets of film material and joining the two sheets along two elongate edges of each sheet to form the handling sleeve. In either of these methods, the sheets of film material may be provided with a point of weakness extending perpendicular to their elongate edges. As such, formation of the handling sleeve can simultaneously form a sleeve attachment portion, separated from the handling sleeve by the point of weakness. The edges of the sheet may be joined by welding; mechanical seal; heat seal; pressure seal; adhesive; solvent bond; ultrasonic weld; laser weld; impulse weld; or friction weld. In a further alternative, the method may comprise attaching a preformed handling sleeve to the funnel in the sheathing station, e.g. by inserting the catheter tube into a preformed sleeve.

Accordingly in one embodiment there is provided a method of processing a catheter assembly comprising a catheter tube coupled to a funnel and a handling sleeve releasably attached to the funnel; the method comprising engaging the funnel, such that the catheter tube hangs down from the funnel; advancing the catheter assembly along rails to a sheathing station and attaching the handling sleeve to the funnel in the sheathing station by wrapping a sheet of film material around the catheter tube and joining opposing elongate edges of the sheet to form the handling sleeve, or sandwiching the catheter tube between two sheets of film material and joining the two sheets along two elongate edges of each sheet to form the handling sleeve; wherein the or each sheet of film material is provided with a point of weakness extending perpendicular to their elongate edges, such that formation of the handling sleeve simultaneously forms a sleeve attachment portion, separated from the handling sleeve by the point of weakness.

The method may comprise permanently attaching the sleeve-attachment portion to the funnel. For example, the method may comprise permanently attaching the sleeve-attachment portion to the sleeve to the funnel by welding or gluing. The handling sleeve may be releaseably attached to the sleeve attachment portion that is permanently attached to the funnel.

The releaseable handling sleeve may be releaseably attached to the sleeve-attachment portion that is permanently attached to the funnel by means of a line of weakness, such as perforations between the handling sleeve and the sleeve attachment portion. Alternatively, the handling sleeve of the sleeve may be releaseably attached to the sleeve-attachment portion that is permanently attached to the funnel by means of a frangible connection. For example, a sleeve may be welded to the funnel, such that the region, e.g. spot where the sleeve is welded to the funnel remains permanently attached to the funnel forming a sleeve attachment portion, but the remainder of the sleeve breaks away from the welded region forming the handling sleeve.

The method may comprise releaseably attaching the entire handling sleeve to the funnel, i.e. attaching the sleeve to the funnel, such that the entire sleeve can be released from the funnel, without leaving any portion attached.

As such, one embodiment provides method of processing a catheter assembly comprising a catheter tube coupled to a funnel and a handling sleeve releasably attached to the funnel; the method comprising engaging the funnel, such that the catheter tube hangs down from the funnel; and releaseably attaching the entire handling sleeve to the funnel, such that the entire sleeve can be released from the funnel, without leaving any portion attached

For example, the handling sleeve may be attached by a tight fit between the inner diameter of the sleeve and the outer diameter of the funnel (e.g. a transition fit or interference fit). In one example, the method comprises heat-shrinking a portion of the handling sleeve around the funnel. In another example, the method comprises tack welding of the film of the handling sleeve to form a tight fit around the funnel. In another example, the sleeve may be tack welded to the funnel-tack welding would form a temporary connection, allowing the sleeve to be released by pulling (this could either result on the entire sleeve coming away at the weld, or as outlined above, with the sleeve breaking off from the weld, leaving the welded part, or “sleeve attachment portion” behind). In another embodiment, the method comprises clipping the sleeve to the funnel. The sleeve may be clipped to the funnel with a separate clip, or with a clip that is integral with the funnel.

In other embodiments, the sleeve, or the sleeve attachment portion may be co-moulded with the funnel. Where the sleeve is co-moulded, it may be frangibly attached to the funnel, or it may be releasably attached to a sleeve attachment portion, which in turn is permanently (or optionally releasably) attached to the funnel.

The method may comprise reorienting the catheter, for example after releaseably attaching the handling sleeve. The catheter may be reoriented from a vertical configuration to a horizontal configuration. The catheter may be reoriented from a vertical configuration to a horizontal configuration by reorienting the suspension means.

The method may comprise coiling the catheter. The step of coiling the catheter may be conducted in the horizontal configuration. The step of coiling the catheter may be conducted by a robot. The coiled catheter may be arranged on a carrier.

The method may comprise introducing the coiled catheter into a pouch. The method may comprise introducing the coiled catheter into a pouch on a carrier. The carrier may be a tray. The method may then comprise withdrawing the carrier (e.g. the tray). Again this may assist in automation. The method may further comprise introducing a fluid reservoir, for example a sachet of wetting fluid, for example water, into the pouch. The method may comprise arranging the fluid reservoir and the coiled catheter on the carrier, then introducing the coiled catheter and the fluid reservoir simultaneously.

The method may comprise introducing the coiled catheter into the pouch through a side or end of the pouch, preferably the base end of the pouch, then sealing the side or end of the pouch. The coiled catheter may be introduced through a side or end that is opposite to a side intended to be opened, which may optionally be provided with an opening mechanism and optionally provided with rescaling means.

Introducing the catheter through an end opposite to that which is intended to be opened and optionally rescaled, means that the pouch can be formed with its opening mechanism (as discussed below) and optional resealing means discussed below, whilst it is empty. Clearly it will be more straightforward to provide these features to an empty pouch.

The method may comprise sealing the pouch after introducing the coiled catheter (and optionally the fluid reservoir). The pouch may be sealed by welding, or adhering. The seal may comprise any one or more of: a weld; mechanical seal; heat seal; pressure seal; adhesive; solvent bond; ultraviolet bond; ultrasonic weld; laser weld; impulse weld; or friction weld. The seal may be at the periphery of the pouch.

The method may comprise forming a join between the two walls of the pouch, such that the join and an internal base of the peripheral seal may define a channel to be filled with fluid prior to withdrawal of the catheter from the pouch. The join may comprise any one or more of: a weld; mechanical seal; heat seal; pressure seal; adhesive; solvent bond; ultrasonic weld; laser weld; impulse weld; or friction weld.

According to a second aspect of the invention, there is provided a catheter assembly manufactured by a process according to the first aspect of the invention, optionally including any optional feature set out above.

According to a third aspect of the invention there is provided a catheter assembly comprising a catheter tube coupled to a funnel, and a handling sleeve; wherein the handling sleeve is formed from a flexible film material and is releasably attached to the funnel.

As outlined above, including a loose sleeve makes manufacture more fiddly, but is common, and attaching a sleeve to an introducer tip has been proposed in the patent literature, but has not found favour in industry (presumably because it adds further complication). On the other hand, releaseably attaching a handling sleeve to a funnel is considered to simplify the manufacturing process.

Of course, any of the optional features of the first aspect of the invention may be included in the third aspect of the invention. Indeed, the catheter assembly of the third aspect of the invention may be manufactured according to the method of the first aspect of the invention (and as such may be a catheter assembly according to the second aspect of the invention). Moreover, any of the optional features set out below may equally be included in the method of the first aspect of the invention. Nonetheless, certain preferred aspects bear repetition.

The handling sleeve may be attached via a sleeve-attachment portion. The handling sleeve (and optionally the sleeve-attachment portion) may be formed of a flexible film. A point of weakness may be provided between the handling sleeve and the sleeve-attachment portion. For example, the handling sleeve (and optionally the sleeve-attachment portion) may be formed of a plastics film. The handling sleeve may be made from any one or more of: polypropylene (PP), low density polyethylene (LDPE), high density polyethylene (HDPE) or biaxially orientated polypropylene (BOPP), for example. The catheter and/or pouch may be made from the same or different materials as the handling sleeve(s) and/or handling sleeve elements. In a preferred embodiments, the catheter is a hydrophilic thermoplastic elastomer (TPE), and the handling sleeve(s) and/or handling sleeve elements are LDPE. The funnel may be polyvinyl chloride (PVC). Thus, the handling sleeve and well as the catheter and funnel can be made from suitable materials.

The handling sleeve is preferably a short sleeve. A short sleeve as defined herein is a sleeve that (when attached) does not extend all the way to the tip of the catheter tube. For example, when attached, the handling sleeve may extend along less than 70%, less than 60%, or less than 50% of the distance to the tip of the catheter tube. A distance of at least 30%, 40% or 50% from the tip of the catheter towards the funnel may be exposed.

As such, one embodiment of the invention provides a catheter assembly comprising a catheter tube coupled to a funnel, and a handling sleeve; wherein the handling sleeve is formed from a flexible film material and is releasably attached to the funnel, wherein the handling sleeve is a short sleeve that, when attached, extends along less than 70% of the distance to the tip of the catheter tube, whereby a distance of at least 30% from the tip of the catheter towards the funnel is exposed.

Another embodiment of the invention provides a catheter assembly comprising a catheter tube coupled to a funnel, and a handling sleeve; wherein the handling sleeve is formed from a flexible film material and is releasably attached to the funnel, wherein the handling sleeve is made from any one or more of: polypropylene (PP), low density polyethylene (LDPE), high density polyethylene (HDPE) or biaxially orientated polypropylene (BOPP) and is a short sleeve that, when attached, extends along less than 70% of the distance to the tip of the catheter tube, whereby a distance of at least 30% from the tip of the catheter towards the funnel is exposed.

Whilst the invention may have application to long sleeves that extend all the way from the funnel to the tip of the catheter tube, the benefits of releasable attachment are primarily concerned with short sleeves, because long sleeves would not actually need to be detached from the funnel in use. On the contrary, in order to best use a short sleeve to guide a catheter tube, a short sleeve needs to be moveable all the way to the tip of the catheter tube, in order to shield the tube from the user's hands, as the user manipulates the tip of the catheter into the urethra.

As used herein, the expression “releaseably attached” means the handling sleeve is attached in such a way that it can be manually released, intact, without requiring any tools (e.g. scissors). Moreover, the manner of detachment is consistent and predictable. This can be achieved for example by perforation; or by spot-welding so as to form a point of weakness around the spot weld, such that the welded sleeve attachment portion only remains attached and the handling sleeve is released intact.

For example, the handling sleeve may be released by the application of a pulling force adequate for easy and comfortable detachment of the sleeve for a user of normal dexterity and a user with limited dexterity. For example the handling sleeve may be released by the application of a pulling force of no more than 5 Newtons; no more than 2 Newtons or no more than 1 Newton.

The handling sleeve may be irreversibly detachable from the funnel.

As used herein, the expression irreversibly detachable means the sleeve cannot be re-attached to the funnel without the use of tools (such as a heat-shrink or plastic welding apparatus, or products, such as glue).

The handling sleeve may be frangibly attached to the funnel. For example, the handling sleeve may be frangibly attached via the sleeve-attachment portion.

The sleeve-attachment portion and the handling sleeve may be separable. The sleeve attachment portion and the handling sleeve may be by breaking the point of weakness between the handling sleeve and the sleeve-attachment portion. The point of weakness may be provided by a plurality of perforations. The perforations may be provided about the circumference of the sleeve.

The perforations may comprise a row of holes extending around the circumference of the handling sleeve. The holes may be separated by a distance that is no more than the size of each hole in a direction parallel to the circumference of the handling sleeve. The holes may each have a width and a length perpendicular to a width and a length of the handling sleeve respectively. The holes may be elongate. The aspect ratio (length:width) of each hole may be at least 1.5:1, 2:1, 3:1, 4:1. The holes may be separated by a distance that is no more than 100%, 90%, 80%, 70%, 60%, 50%, 40%, 30%, 20% or 10% the length of each hole. The separation of adjacent holes may depend on the length of the holes, alternatively the separation of adjacent holes may be constant irrespective of the length of the holes. The holes may be separated by a distance that is less than 4 mm, 2 mm or 1 mm. The holes may be separated by a distance that is at least 0.4 mm, 1 mm, or 2 mm. The length of each hole may be at least 0.5 mm, 1 mm, or 2 mm. The length of each hole may be less than 6 mm, 2 mm, 1 mm, or 0.5 mm. Preferably, the length is 2 mm, and the holes are separated by a distance of 1 mm. As such, the perforations provide for a point of weakness in the handling sleeve that is sufficient to allow easy separation of the handling sleeve from the sleeve-attachment portion.

Each hole forming the perforations may be a slit. Where each hole is a slit, it may be formed by a one dimensional cut in the sleeve. Where each hole is a slit it may have an aspect ratio (length:width) that is effectively 1:0. Each cut may be made over a distance corresponding to the desired length of each hole. Alternatively, the slitted perforations may be formed in any other suitable way. Advantageously, where the holes are slits, the catheter is less likely to be exposed through the handling sleeve than if the hole was not a slit. This helps prevent inadvertent contamination of the catheter while handling.

The point of weakness may be provided by a tear-off strip. The point of weakness may be provided by a peel-off adhesive. Thus, the point of weakness may be configured in a variety of different ways.

The separation force required to detach the handling sleeve from the sleeve attachment portion may be less than the separation force required to detach the sleeve attachment portion from the funnel.

The sleeve attachment portion may be adhered or welded to the funnel.

The catheter assembly may be provided in a pouch.

The handling sleeve may have a length measured parallel to the axis of the catheter, and a width measured perpendicular to the axis of the catheter. The width of each handling sleeve may be constant along at least part of its length. The width of the handling sleeve may vary along at least part of its length. The length of the handling sleeve may be less than: 275 mm; 250 mm; 200 mm; 150 mm; 125 mm; or 100 mm. The length of a handling sleeve may be at least: 50 mm; 100 mm; 125 mm; 150 mm; 200 mm; or 250 mm. Preferably, the length of the handling sleeve may be between 120-100 mm, more preferably 110 mm. The width of the handling sleeve may be less than: 60 mm; 40 mm; 30 mm; 25 mm; 20 mm; or 15 mm. The width of a handling sleeve may be at least: 10 mm; 15 mm; 20 mm; 25 mm; 30 mm; or 40 mm. Preferably, the width of the handling sleeve is between 15-25 mm.

Where the width of a handling sleeve is constant along at least part of its length, it may be formed from two rectangular strips of material. In one example, the rectangular strips of material are 110 mm in length and 19 mm wide. At least part of a handling sleeve may be conical. The width of a conical part may increase or decrease along the length of the conical part of the handling sleeve. A conical part of a handling sleeve may be formed from strips of material that are isosceles trapeziums.

The catheter may be a urinary catheter. The catheter may be a male urinary catheter. The catheter may be an intermittent catheter. In one embodiment, the catheter is an intermittent male urinary catheter. Thus, the features of the present invention allow intermittent male urinary catheters to be provided in a compact packaging that also facilitates convenient and easy withdrawal, use and re-packaging of the catheter.

The catheter may comprise a tip at proximal end for insertion into the body and the funnel at a distal end. The catheter assembly may be arranged in the pouch such that the funnel may be withdrawn from the pouch before the proximal end. Thus, the handling sleeve can be arranged such that they can be used to withdraw the catheter from the pouch, this helps ensure the catheter remains clean and sterile prior to use and reduces the risk of infection.

The catheter assembly may have no introducer tip. At least some users prefer catheter assemblies without introducer tips.

The handling sleeve may be movable along the length of the catheter.

The handling sleeve may be configured to enable arrangement of the catheter within the pouch after use. The handling sleeve may be arrangeable at the proximal end of the catheter after use. As such, the catheter may be handled at one end with the funnel (which will remain clean on the outside) and at the other end with the handling sleeve.

At least part of the catheter may be configured to be wetted during withdrawal of the catheter from the pouch. At least part of the handling sleeve may be configured not to be wetted during withdrawal of the catheter from the pouch. Less than 50%, 40%, 30%, 20%, or 10% of the length of a handling sleeve may be configured to be wetted during withdrawal of the catheter from the pouch. Thus, the catheter may be adequately lubricated for use without significantly affecting the handling sleeve.

The handling sleeve may be impermeable to fluids.

The pouch may comprise two walls and a peripheral seal between the two walls. The catheter may be arranged within the pouch in a curved configuration. A join may be provided between the two walls of the pouch. The join and an internal base of the peripheral seal may define a channel to be filled with fluid prior to withdrawal of the catheter from the pouch. The pouch and catheter may be arranged such that during withdrawal of the catheter from the pouch, the distal end of the catheter is withdrawn from the pouch before the proximal end and the proximal end of the catheter passes through the channel. The handling sleeve may be arranged not to pass through the channel during withdrawal of the catheter from the pouch. Thus, the catheter may be removed and simultaneously lubricated without the handling sleeve passing through the channel and becoming wet/lubricated.

The handling sleeve may be arranged around a section of the catheter that is not intended for insertion into the body. The handling sleeve may not be arranged around sections of the catheter intended for insertion into the body. Sections of the catheter not intended for insertion into the body may not be arranged to pass through the channel during withdrawal of the catheter from the pouch. Sections of the catheter intended for insertion into the body may be arranged to pass through the channel during withdrawal of the catheter from the pouch. Thus, the one or more handling sleeves do not inhibit the lubrication of the catheter whilst it passes through the channel during withdrawal of the pouch.

The pouch may comprise a fluid reservoir. The fluid reservoir may be configured to release fluid into the pouch to form a pool of fluid in the channel. The volume of the fluid reservoir is preferably sufficient to completely fill the channel, i.e. with the pouch held with its base lowermost, the fluid may extend to at least the level of the bottom of the join, so that the fill level of the channel is 100% or more, for example at least 150%, at least 200% or even at least 300%. Alternatively, the fill level of the channel may be: at least 70%, at least 80%, at least 90%; or at least 95% the volume of the channel. At least one handling sleeve element may be arranged above the fill level of the channel prior to withdrawal of the catheter from the pouch. Thus, the present invention provides for an assembly with a reservoir that is able to deliver fluid into the channel. The size of the reservoir is selected to ensure sufficient fluid is delivered and the catheter can be adequately lubricated on removal, in addition, at least one handling sleeve element remains above the fill level and remains in a dry state enabling more convenient handling of the catheter.

The pouch may be sealed and sterilized. The fluid reservoir may be arranged within the (scaled and sterilized) pouch. The fluid reservoir may be provided as a separate device, or incorporated into the pouch material. The fluid reservoir may comprise a deformable, frangible or burstable sachet. Thus the fluid reservoir is conveniently located within the pouch where it may directly release fluid into the channel formed by the join and the base of the peripheral seal. A deformable, frangible or burstable reservoir enhances the convenience for a user as fluid can be easily released without additional tools and before the pouch is unsealed, thus reducing the chances of accidental wetting of the user. A sachet in particular can be easily handled on an assembly line and introduced more simply than attempting to handle fluid, so as to it into the package during manufacture.

The catheter may be arranged between the join and the internal base of the peripheral seal. The join may be separate from the base of the peripheral seal. The channel may be an open-ended channel. Thus, the catheter is held within the pouch in the open-ended channel formed by the join and the internal base of the peripheral seal. As such, when withdrawn, the catheter is drawn through the channel and is adequately lubricated.

The join and the peripheral seal may be independent. The join may be provided in a spot. The join is preferably provided by a weld, e.g. a spot weld. Such a weld may be provided after the catheter (and optionally the sachet) have been arranged between the walls, and can be achieved from outside of the pouch. Alternatively, the join may be provided by any of a: mechanical seal; heat seal; pressure seal; adhesive; solvent bond; ultrasonic weld; laser weld; impulse weld; or friction weld. Similarly, the peripheral seal be provided by any one or more of a weld; mechanical seal; heat seal; pressure seal; adhesive; solvent bond; ultrasonic weld; laser weld; impulse weld; or friction weld. At least part of the peripheral seal may be formed by a fold. In such embodiments, a single sheet may be folded, for example at the base or one of two sides, to form two opposing walls of the pouch. The join can be provided in a single location allowing the peripheral seal and join to be formed separately. Alternatively, the weld forming the join may be provided at the same time as the peripheral seal. In some embodiments, the join and peripheral seal are a single continuous seal. The manufacturing process of the join can thus be very convenient and avoid major modification of a typical packaged catheter assembly production line.

The catheter may comprise a surface which is activated by the fluid in the channel. For example, the catheter may comprise a hydrophilic surface, formed for example by a hydrophilic coating. Where a hydrophilic surface is provided, the fluid may be polar or water-based. The catheter may comprise a hydrophobic surface, which is activated by the fluid in the channel. For example the catheter may comprise a hydrophobic coating. Where a hydrophobic coating is used, the fluid may be non-polar. Thus the catheter is easily lubricated by fluid in the channel and is easy and comfortable to use.

The handling sleeve may comprise a surface which is not activated by the fluid. A surface of the handling sleeve may be hydrophobic. An inner surface of the handling sleeve may be hydrophobic. The handling sleeve may be embossed with a pattern. An inner surface of the handling sleeve may be embossed with a pattern. The pattern may be a linen pattern. Preferably an inner surface of the handling sleeve adjacent to the catheter in use is hydrophobic and embossed with a pattern. Advantageously, this reduces friction between the handling sleeve and the catheter in use. In some embodiments, the surface properties of parts of the handling sleeve, or individual handling sleeve elements may vary depending on their functionality. For example, where a part of the handling sleeve, or a handling sleeve element, is intended not to move along the length of the catheter, its surface properties may be selected to increase friction between the handling sleeve and the catheter. Thus, the surface properties of the handling sleeve can be selected to ensure the handling sleeve and catheter are easy to use.

The pouch may comprise an interaction region defining a first point of contact for a user of the packaged catheter assembly. The interaction region may facilitate user access to the contents of the pouch. Actuation of the interaction region may create an opening in or near an upper edge of the peripheral seal. The catheter may be withdrawn through the opening. Thus, access to the pouch is provided at or near an upper edge of the pouch and as such, the fluid in the channel (at the bottom of the pouch) is retained in the pouch while the catheter is withdrawn reducing the risk of spilling fluid outside the pouch.

The interaction region may comprise a breakable region of the peripheral seal. The method may comprise forming a breakable region in the peripheral seal. The breakable region may comprise a tear-away region which may comprise a tear line defining a line along which the pouch may be torn to at least partially separate the tear-away region from the remainder of the pouch, thereby creating an opening in the peripheral seal. The tear-away region may comprise a tear start. The tear start may be provided at a first end of the tear away region. The tear start may comprise a notch, or area of weakness, in one or both of the walls of the pouch. The tear start may define the first part of the pouch to be torn. The tear-away region may comprise a tear stop. The tear stop may be provided at a second end of the tear-away region. The tear stop may act to prevent or reduce the likelihood of the tear-away region being completely separated from the remainder of the packaging. The tear-away region may be substantially triangular in shape having a sloped upper edge. Thus the breakable region provides a convenient and easy means to open the pouch with minimal effort and no additional equipment/tools.

The interaction region may comprise a sealing arrangement. The sealing arrangement may comprise one or more sealing members operable to provide a user-scalable seal between the two walls of the pouch, so as to close the pouch. In one embodiment, first and second sealing members are provided on an interior surface of the two walls of the pouch. The scaling arrangement may provide a watertight and/or fluid impermeable seal when closed. Thus the pouch can be rescaled to ensure the fluid in the channel is not subsequently released from the pouch after use. This also allows the catheter to be repackaged back into the pouch after use and retained within it.

The sealing arrangement may come pre-sealed. In such embodiments, user access to the contents of the pouch may be provided by opening the sealing arrangement. The scaling arrangement may be resealable. The sealing arrangement may be any of: a zip-lock seal; hook and loop seal; hook and hook seal; or adhesive layer. Therefore, the sealing arrangement can be used to ensure the contents of the pouch remain inside until the scaling arrangement is opened, and the pouch can still be rescaled after use. This can help if the breakable region requires force to be applied to the pouch to be opened, as the pouch may move or jerk in position while being opened causing fluid or the catheter to exit the pouch.

The interaction region may comprise a breakable region and a scaling arrangement. In certain embodiments, the interaction region comprises a tear-away region corresponding to the upper edge of the peripheral seal, and a resealable arrangement below the tear-away region.

The peripheral seal may have an upper edge. The peripheral seal may be substantially rectangular in shape with two lateral edges between the base and upper edge. The peripheral seal may have a height from the base to the upper edge of at least 10 cm, 15 cm, or 20 cm; and/or a height of no more than 10 cm, 15 cm, 20 cm or 25 cm, such as between 15 cm and 20 cm. The peripheral seal may have a width between the lateral edges of at least 5 cm, 10 cm; or 15 cm; and/or a width of no more than 10 cm or 15 cm, or 20 cm such as between 5 cm and 15 cm. The catheter may have a length of at least: 20 cm; 25 cm; 30 cm; 35 cm; 40 cm; 45 cm; or 50 cm, for example between 35 and 45 cm. In one embodiment, the peripheral seal has a height 10-25 cm and a width of 6-11 cm, and the catheter has a length of at least 30 cm. Thus, the peripheral seal defines a “pocket-sized” pouch that can store a catheter within in a curved configuration to reduce the apparent size of the catheter and enable convenient and discreet storage.

The funnel may be arranged closer to an upper edge of the peripheral seal than the base of the peripheral seal. The funnel may be arranged in the upper half of the pouch, or the upper quarter of the pouch. The funnel may be arranged above the join. The funnel may be arranged above the fill level of the fluid. Thus, the funnel is easy to access and presents a handling sleeve to the user so that the catheter may be withdrawn conveniently using the handling sleeve. The handling sleeve is also positioned so as not to be pulled through the fluid, so will not be wetted and will remain easy to handle.

The handling sleeve may be arranged closer to an upper edge of the peripheral seal than the base of the peripheral seal. At least part of the handling sleeve may be arranged in the upper half of the pouch or the upper quarter of the pouch. At least part of the handling sleeve may be arranged above the join. At least part of the handling sleeve may be arranged above the fill level of the fluid. Thus, the handling sleeve is easy to access so that it may be withdrawn before the proximal end of the catheter as required by the invention. It is also positioned so as not to be pulled through the fluid, so at least part of it will not be wetted and will remain easy to handle, providing a dry surface that can be gripped and used to manoeuvre the catheter.

The join may be provided at a point equidistant from each lateral edge. Alternatively it may be offset. The method may comprise providing the join at a point equidistant from each lateral edge. Alternatively it may comprise proving the join in an offset lateral position. For example it may be arranged anywhere between 25% and 75% of the width of the pouch. The distance between the left and right lateral edges of the peripheral seal may be the width of the pouch. The distal end of the catheter may be provided at a distance from one left edge that is no more than 20%, 10%, 5% or 1% of the width of the pouch. Thus the join and distal end are provided in locations that make the catheter easy to withdraw and lubricate without spilling lubricant. The provision of a join that is equidistant between the edges ensures the channel volume is restricted and that the fluid level within it is optimised for lubricating the catheter.

The funnel may be arranged closer to one (e.g. a left) lateral edge of the peripheral seal than another (e.g. a right) lateral edge of the peripheral seal. In embodiments comprising an interaction region, actuation of the interaction region may preferentially first create an opening in a first (e.g. left) lateral edge of the peripheral seal. In such embodiments, the funnel may be arranged closer to the first (e.g. left) lateral edge. In embodiments comprising a tear-away region and a tear start, the location of the tear start may correspond to a lateral edge of the peripheral seal. In such embodiments, the funnel may be arranged closer to the lateral edge of the peripheral seal corresponding to the location of the tear start. Thus, the funnel and handling sleeve are located next to the first part of the pouch to be opened and is easier to withdraw.

The join may be provided closer to the base of the peripheral seal than an upper edge of the peripheral seal. The distance between the base and the upper edge of the peripheral seal may define the height of the pouch. The distance between the join and base may be at least: 10%; 15%; 20%; 25%; 30%; 35%; 40% or 45% of the height of the pouch. The distance between the join and base may be less than: 50%; 45%; 40%; 35%; 30%; 25%; 20%; or 15% of the height of the pouch. In particular, the join can be arranged at between 20% and 30% of the height of the pouch. The join may be circular. The join may have a radius equivalent less than 15%; 10%; 5%; or 1% the height of the pouch. In a preferred embodiment, the join has a radius of 5% the height of the pouch. Thus, the join can be arranged to ensure the catheter is adequately lubricated and that it defines a channel of suitable size to allow easy withdrawal of the catheter without excessive bending of the catheter or excessive force to be applied.

In one embodiment, the join is provided at a point equidistant from each lateral edge of the pouch and at a distance from the base that is 20-30% of the height of the pouch.

The funnel may be arranged so as not to pass through the channel during withdrawal of the catheter from the pouch. The handling sleeve may also be arranged so as not to pass through the channel during withdrawal of the catheter. The handling sleeve may be no more than: 25%; 20%; 15%; 10%; or 5% of the length of the catheter, but could be longer. This makes the catheter more convenient to use as the fluid is only applied to the necessary parts of the catheter. It also facilitates using less fluid as it is applied in a targeted manner to the catheter.

The angle subtended by the ends of the catheter when curved in the pouch may be at least: 180; 210; 240; 270; 300; 330; or 360 degrees; it may even be at least 420; 480 or 540 degrees. It may be less than 720; 630 or 540 degrees. The catheter may be arranged within the pouch in a curled or coiled configuration either before use, after use, or both before and after use. Prior to use, the catheter may be arranged such that the join is within an area defined by the curl or coil of the catheter. After use, the catheter may be arranged such that the join is not be provided within an area defined by the curl or coil of the catheter. The peripheral seal may confine the catheter in its curved configuration. As such, the catheter is curved, curved or coiled to reduce the size of the pouch and ensure that long catheters can be conveniently and discreetly stored ready for use.

The catheter may be arranged such that during withdrawal at least: 30%; 40%; 50%; 60%; 70% or 80% of the length of the catheter passes through the channel. In one embodiment, 70-80% of the length of the catheter passes through the channel during withdrawal. Advantageously, only the part of the catheter intended to enter the body is lubricated and therefore fluid is not wasted and the catheter is easier to use.

The walls of the pouch may be formed of any suitable material, such as film materials. The walls may be impermeable to fluids. The walls may be made of a plastics material. The walls may be made from any one or more of: polypropylene (PP), polyethylene terephthalate (PET), low density polyethylene (LDPE), metalized polyester (MET PET) or orientated polypropylene (OPP), for example. The walls may comprise a laminate of two or more layers. Each wall may comprise an inner layer and an outer layer. Each wall may comprise inner and outer layers of polypropylene. Each wall may comprise an inner layer of orientated polypropylene and an outer layer of polypropylene. Each wall may comprise an inner layer of polypropylene and an outer layer of polyethylene, for example. The inner layer and outer layer may have the same thickness. In other embodiments, the inner layer and outer layer may have different thicknesses. The inner layer may have a smaller thickness than the outer layer. In embodiments, the inner layer may have a thickness of between 5-20 microns, or between 10-10 microns, or between 10-15 microns, and the outer layer may have a thickness of between 10-50 microns, or between 20-40 microns, or between 35-45 microns. In one example, the inner layer may have a thickness of approximately 12 microns and the outer layer may have a thickness of approximately 40 microns.

The pouch may be an opaque pouch. The pouch may comprise a transparent or translucent window (preferably corresponding to the position of the fluid reservoir in the pouch and/or a wetting region into which fluid is intended to flow from the fluid reservoir). The catheter and/or one or more handling sleeves may be arranged within the pouch so as to be hidden from view. A fluid reservoir may be provided within the pouch, and the pouch may be configured so as to allow a user to view the fluid reservoir and/or view fluid released from the fluid reservoir.

Thus, the present invention provides for a packaged catheter assembly that is discreet as the contents of the assembly are hidden. The window provides a visual guide so that during use of the catheter, the user can easily identify the fluid reservoir, and/or easily identify that fluid has been successfully released from the reservoir into a wetting region of the pouch to lubricate the catheter prior to use. Whilst the provision of a transparent/translucent window is the preferred approach for allowing a user to view whether fluid has been successfully released from the reservoir, alternative approaches are considered feasible. For example, rather than providing a transparent/translucent window in an opaque wall of the pouch, it is conceived that the functionality could be achieved by providing a transparent/translucent pouch wall, but including an intervening opaque wall, within the pouch provided between the transparent/translucent pouch wall and the catheter (and optionally the reservoir). The intervening wall could include a window, or fluid released from the reservoir could flow between the outer transparent/translucent wall and the intervening wall, providing an indication of successful release.

The pouch may comprise two opaque walls. The window may be provided in one of the opaque walls. The window may be spaced from at least one edge of the pouch or the wall it is provided in. The window may be spaced from all edges of the pouch. The window may be less than: 25%; 20%; 15%; 10% or 5% the total area of the pouch and/or the wall it is provided in. The window may correspond to the size and/or shape of the fluid reservoir. The window may be a smaller size than the fluid reservoir. In one embodiment, the window is the same shape but a smaller size than the fluid reservoir. Thus, in embodiments of the invention, the window advantageously corresponds to the size, shape and position of the fluid reservoir and is provided in a discrete manner, separated from the edges of the pouch and only provided in a section of the pouch's wall.

The window may correspond to the position of an indicator region of the pouch. the indicator region may correspond to a wetting region, which may extend from the base of the pouch to a level at which sufficient fluid has escaped from the pouch to wet the catheter. The indicator region may be configured to indicate successful release of fluid from the reservoir. The indicator region may comprise a fill-level marker indicating sufficient fluid is in the wetting region. Thus the window also provides a visual guide to ensure fluid released from the reservoir has been delivered to a specific area of the pouch (e.g. a channel) or that it has successfully lubricated the catheter.

The pouch may be substantially rectangular in shape with two lateral edges, a base, and an upper edge. The height of the pouch may be defined as the distance between the base and upper edge. The width of the pouch may be defined as the distance between the lateral edges of the pouch. The window may span at least: 20%, 30%; 40%; 50%; 60%; 70%; 80% or 90% the height of the pouch. In one embodiment, the window spans at least 30-40% of the height of the pouch and the window is between 15-20% of the width of the pouch. Thus, the fluid reservoir is easily visible as the window is aligned with it, but simultaneously, the area of the window is minimised to ensure the contents of the pouch remain generally not visible, on account of the fact that but for the window, the pouch is opaque.

The catheter in the pouch is preferably hidden. The catheter may be hidden by the opaque walls of the pouch. The catheter may be hidden by the fluid reservoir. The one or more handling sleeves in the pouch is preferably hidden. The one or more handling sleeves may be hidden by the opaque walls of the pouch. The one or more handling sleeves may be hidden by the fluid reservoir. This is particularly advantageous from the point of view of discretion—the catheter and handling sleeve(s) are likely to appear to observers as some kind of medical device, even if they are not aware of its precise nature; hiding it, either by ensuring that it is arranged out of register with the window, or by hiding it behind another object, for example the fluid reservoir (but optionally a dedicated sheet material, for example) obscures this.

Notably, the fluid reservoir (or other obscuring object) may be opaque, and may have a colour that corresponds to, or contrasts with that of the walls of the pouch. For example, the reservoir (or other obscuring object) may be in the same colour, but a different shade to that of the pouch, or where the opaque pouch has multiple colours, may be a shade of one of those colours. A grey or silver is considered particularly preferable for the reservoir (or other obscuring object) from the point of view of discretion.

The handling sleeve may be opaque and have a colour that corresponds to, or contrasts with, that of the walls of the pouch and/or the catheter. Thus, the handling sleeve can be constructed to be similar to the colour of the pouch and/or catheter and thereby provide a more discreet catheter.

The window may be patterned; i.e. at least part of the window may be provided with a pattern thereon, such as a pattern formed by opaque regions, such as spots, stripes or the like. The pattern may provide a graduated appearance to the window. The pattern may provide a graduated appearance to one or more of the edges the window. The pattern may blend the window into the surrounding packaging of the pouch. Thus, the window is less obvious to the untrained eye and is therefore the packaged catheter assembly (both before and after use) is more discreet for the user to carry with them.

According to a fourth aspect of the present invention there is provided a method of handling a catheter, wherein the catheter comprises at least one handling sleeve releasably attached to a funnel, the method comprising releasing the handling sleeve from the funnel and manipulating the catheter using the handling sleeve.

The catheter assembly may be a catheter assembly according to the second or third aspect set out above, optionally including any of the optional features set out above. Indeed, it may comprise any of the optional features without necessarily including all the features set out in the second or third aspects above.

The method may comprise moving the handling sleeve along the length of the catheter. Thus, the handling sleeve may be easily arranged to assist handling different parts of the catheter.

The catheter may comprise a proximal end for insertion into the body and a distal end. The method may comprise arranging the handling sleeve at or near to the proximal end of the catheter. Thus, the ends of the catheter can be conveniently handled without touching the used catheter surface itself.

The method may comprise forming a knot in the catheter. The method may comprise forming a loop in the catheter using the handling sleeve and the funnel. The method may comprise passing an end of the catheter through the loop so as to form a knot. Preferably, the distal end may be passed through the loop. Thus, the size/shape of the catheter may be further restricted by knotting the looped catheter. In addition, both ends of the catheter are retained in position ensuring the catheter is easily repackaged in the pouch.

The method may comprise inserting the proximal end into the funnel. The method may comprise compressing the funnel to retain the proximal end within it. Thus, the catheter's ends are connected together to form a circular shape which makes the catheter easier to handle and package.

The method may comprise introducing the catheter to the body and withdrawing the catheter from the body using the handling sleeve of the catheter.

The method may comprise opening a pouch and withdrawing the catheter from the pouch.

Where the catheter is a urinary catheter, the method may further comprise introducing the catheter by its proximal end into the urethra. The method may comprise releasing urine through a funnel at the distal end of the catheter. The method may further comprise removing the catheter.

### DETAILED DESCRIPTION OF THE INVENTION

In order that the invention may be more clearly understood one or more embodiments thereof will now be described, by way of example only, with reference to the accompanying drawings, of which:

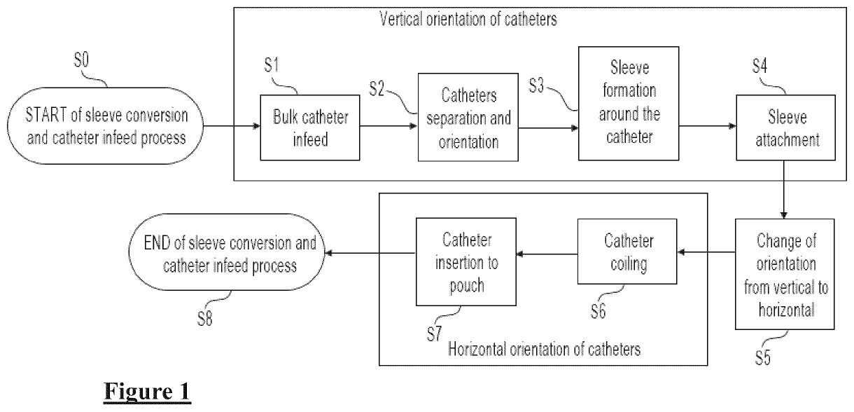

FIG. 1 is a flow chart showing a process according to the invention;

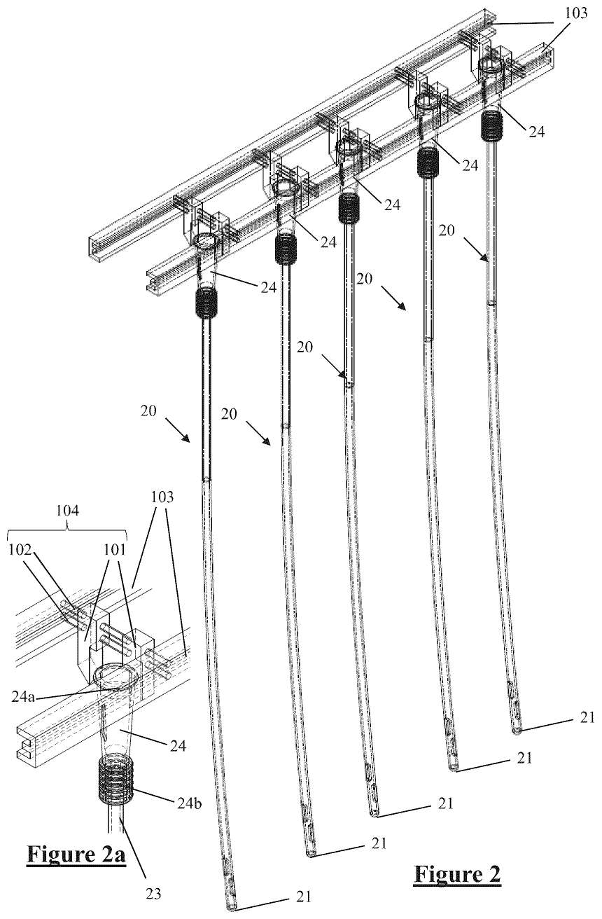

FIG. 2 is an isometric view of a series of catheter assemblies being processed in accordance with the method of the invention;

FIG. 2a is an enlarged view of the top of a catheter assembly in the process of FIG. 2;

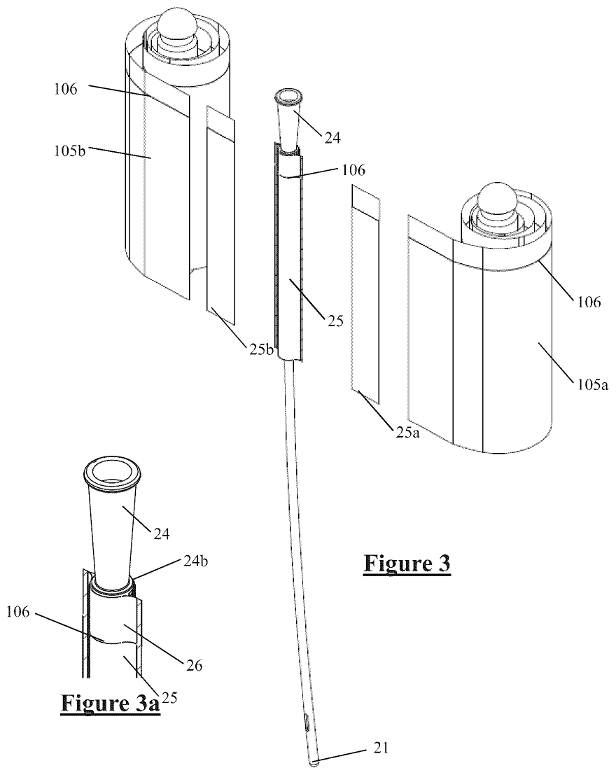

FIG. 3 is a schematic view of the formation of a sleeve around the funnel of a catheter in the method of the invention;

FIG. 3a is an enlarged view of the top of a catheter assembly in the process of FIG. 3;

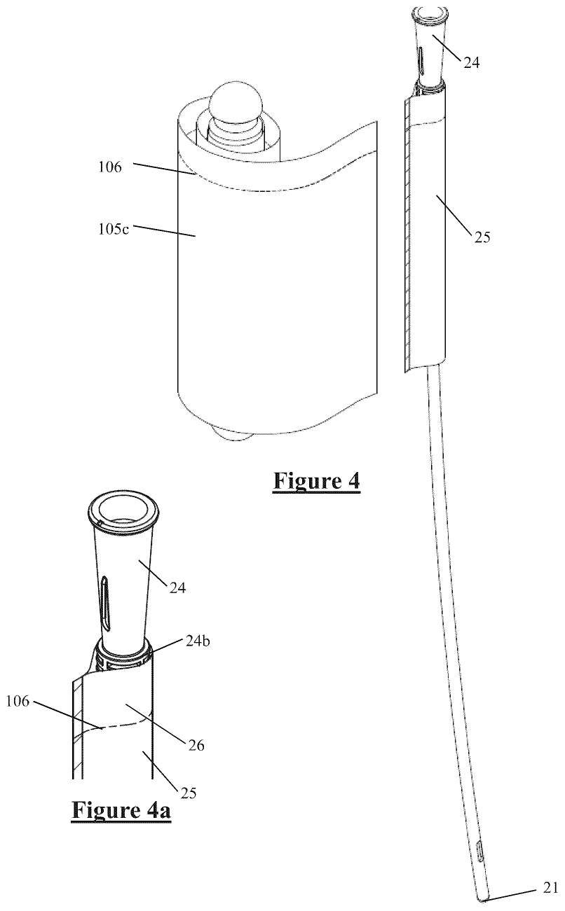

FIG. 4 shows an alternative embodiment of the formation of a sleeve around the funnel of a catheter in the method of the invention;

FIG. 4a is an enlarged view of the top of a catheter assembly in the process of FIG. 4;

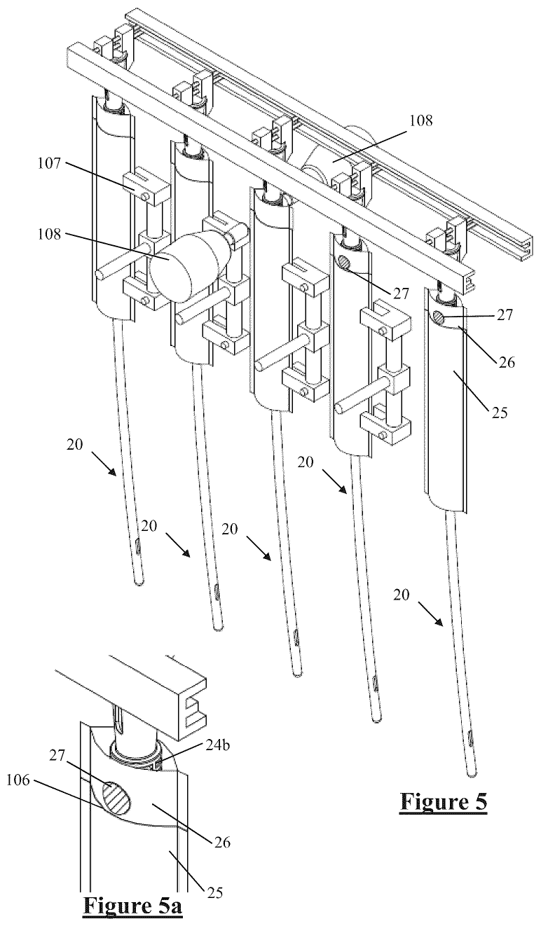

FIG. 5 shows an isometric view of one method of attaching the sleeve to the funnel in accordance with the method of the invention;

FIG. 5a is an enlarged view of the top of a catheter assembly in the process of FIG. 5;

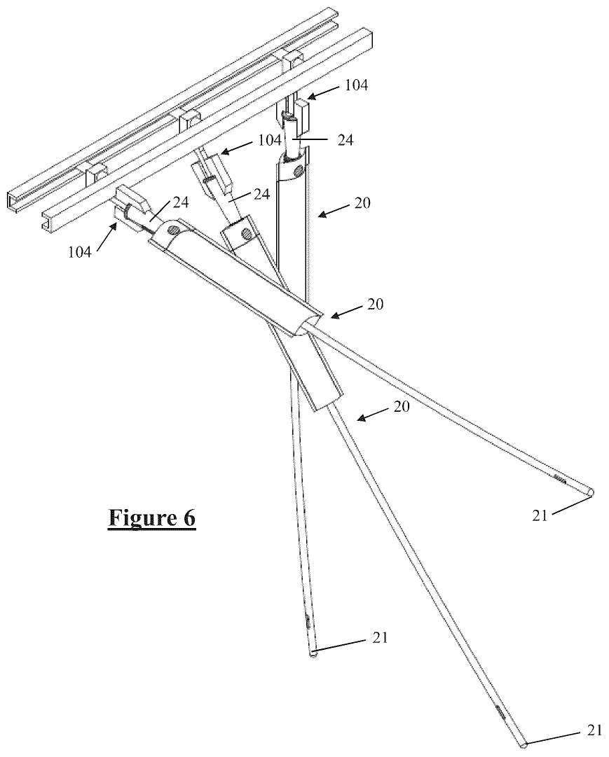

FIG. 6 shows an isometric view of the reorientation of the catheter assemblies in the method of FIG. 1;

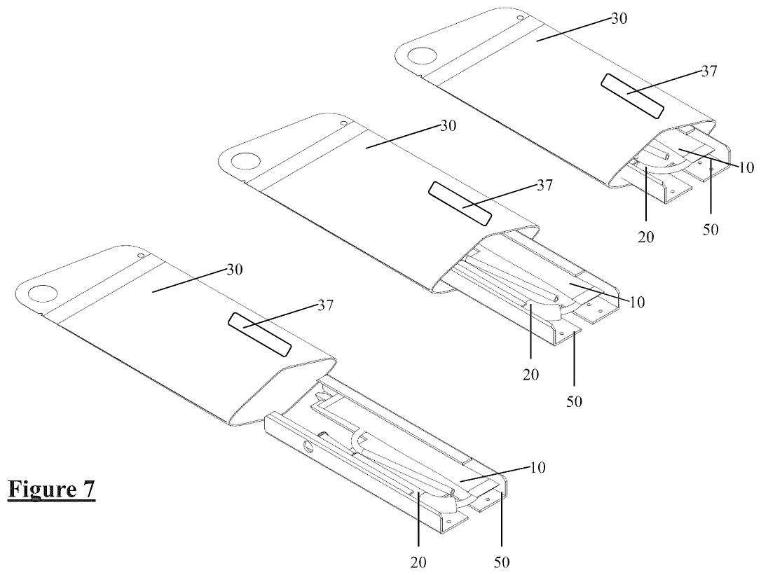

FIG. 7 shows a simplified isometric view of the introduction of the catheters into respective pouches;

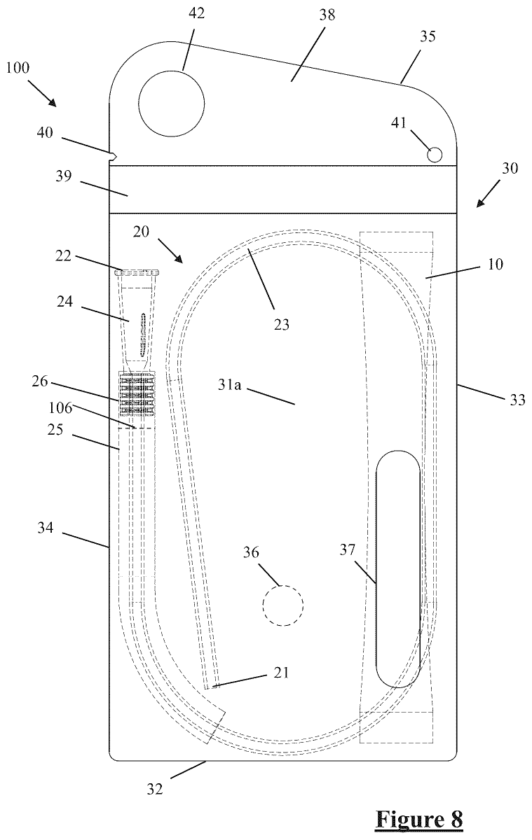

FIG. 8 is a front view of a packaged catheter assembly;

FIG. 9 is a front view of the packaged catheter assembly of FIG. 8 where fluid has been released into the pouch and the package has been opened;

FIG. 10 is a front view of the packaged catheter assembly of FIGS. 8 and 9 where the catheter has been almost fully withdrawn from the pouch;

FIG. 11 is a cross sectional view of the catheter assembly of FIGS. 8-10 removed from the pouch;

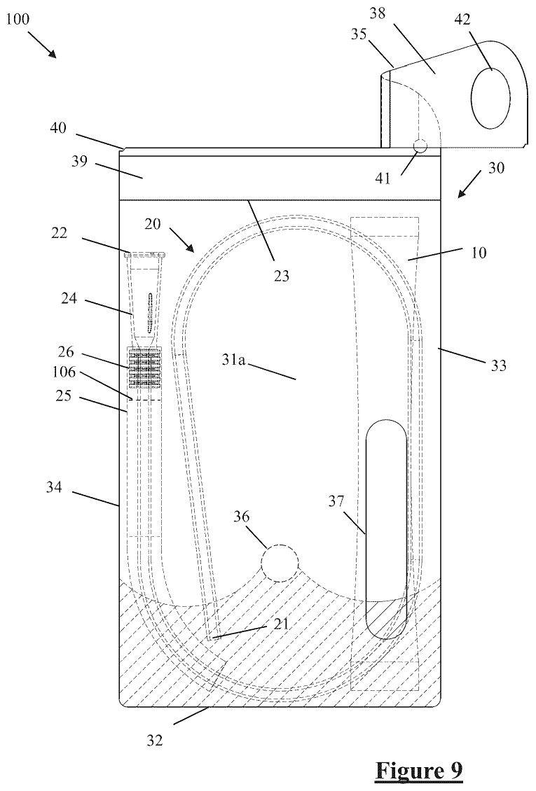

FIG. 12 is a cross sectional view of the catheter assembly of FIG. 11 with the sleeve detached;

FIG. 13 is a front view of a third embodiment of a catheter assembly manufactured in accordance with the invention, where the catheter has been almost fully withdrawn from the pouch;

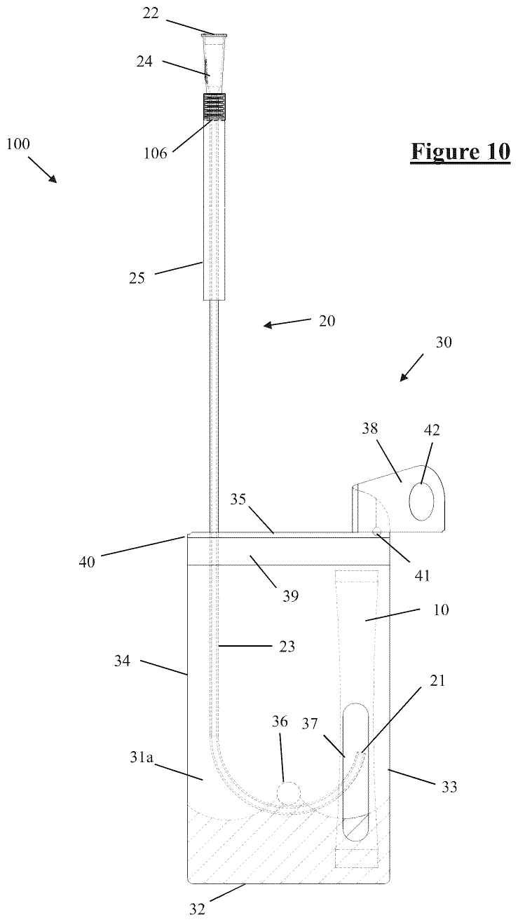

FIG. 14 is a front view of an alternative method of arranging a sleeve around the funnel of a catheter assembly in accordance with the invention;

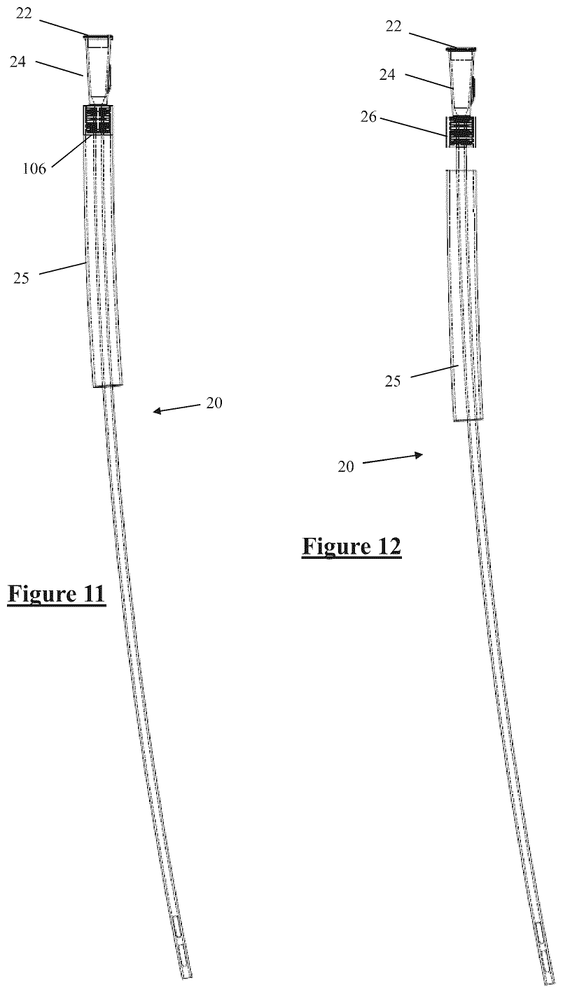

FIG. 15 is a front view of a fourth embodiment of a catheter assembly manufactured in accordance with the invention;

FIG. 16 is a cross sectional view of a fifth embodiment of a catheter assembly manufactured in accordance with the invention;

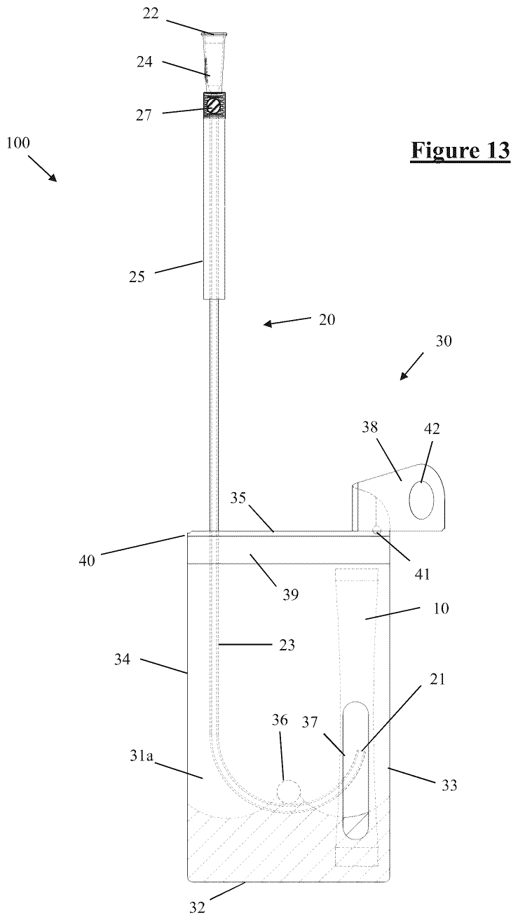

FIG. 17 is a cross sectional view of a sixth embodiment of a catheter assembly manufactured in accordance with the invention;

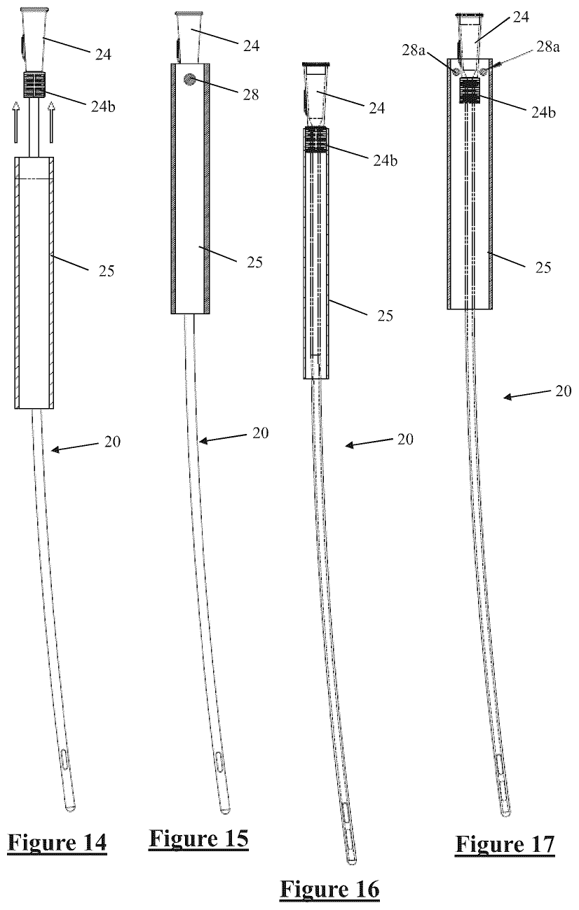

FIG. 18 is a cross sectional view of a seventh embodiment of a catheter assembly manufactured in accordance with the invention;

FIG. 18A is an enlarged cross sectional view of the catheter of FIG. 18;

FIG. 19 is a cross sectional view of an eighth embodiment of a catheter assembly manufactured in accordance with the invention;

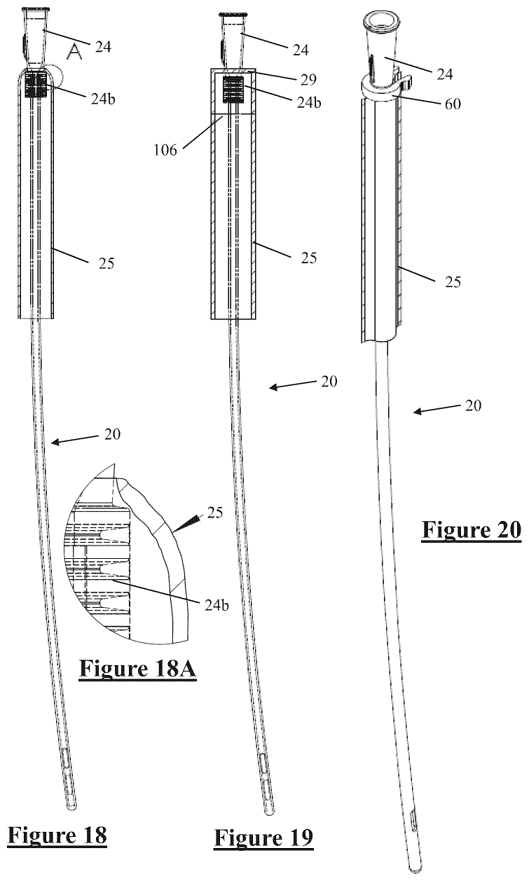

FIG. 20 is a perspective view of a nineth embodiment of a catheter assembly manufactured in accordance with the invention;

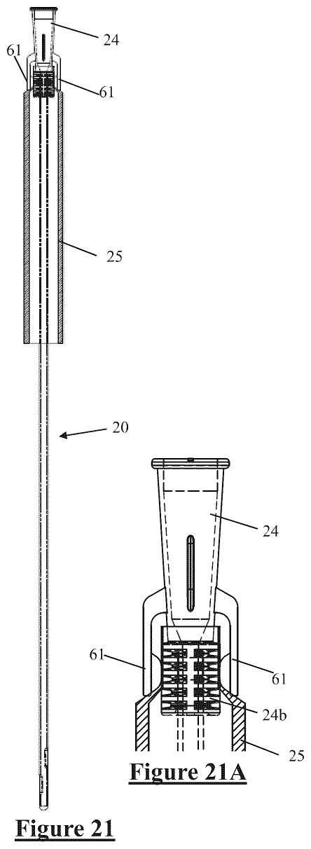

FIG. 21 is a cross sectional view of a tenth embodiment of a catheter assembly manufactured in accordance with the invention; and

FIG. 21A is an enlarged cross sectional view of the catheter of FIG. 21.

In the figures, as is conventional, broken lines show hidden features.

The present disclosure concerns a catheter/catheter assembly and in particular a handling sleeve for the catheter. It is described in the context of a packaged catheter assembly. The packaged catheter assembly comprises a pouch and the catheter assembly. The catheter is arranged within the pouch. The catheter comprises a handling sleeve which is releasably attached to the funnel, so as to be detachable from the funnel for use in manipulating the catheter, after withdrawal of the catheter from the pouch.

This arrangement with the handling sleeve attached to the funnel has benefits in the processing of the catheter assembly during manufacture and assembly.

Referring to FIGS. 1-8, a method of producing a first and second embodiment of a packaged catheter assembly 100 is shown.

The packaged catheter assembly 100 (best seen in FIG. 8) comprises a fluid reservoir 10, a male intermittent single-use urinary catheter 20, and a pouch 30.

With reference to FIG. 1, the process of the invention for manufacturing the packaged catheter assembly 100 is concerned with sleeve conversion, i.e. the conversion of film material into sleeves (25) and catheter infeed, i.e. the feeding of catheter assemblies 20 into the pouches 30. Step S0 represents the start of the process. At this stage, catheters have already been produced in bulk and each catheter 20 comprises a proximal end 21 for insertion into the body, a distal end 22, and a flexible tube 23 connecting the two ends 21, 22. The distal end 22 comprises a funnel 24 to guide urine from the bladder out of the tube 23 in use.

At step S1, the catheters 20 are fed in bulk to a hopper (not shown) or the like. Throughout steps S1 to S4, the catheters 20 are in a vertical orientation. This hopper therefore holds the catheters upright, with their funnels at the top, and at step S2, the catheters are separated and oriented by picking them by their funnels, such that the catheter tube 23 hangs from the funnel 24, as shown in FIG. 2. As best seen in FIG. 2a, each funnel has a rim 24a at the distal end, and this rim 24a is gripped by a pair of gripping arms 101. The gripping arms 101 are connected, via a pair of laterally extending pins 102, which connect the arms 101 and extend through them, to a pair of processing rails 103. The gripping arms 101 and their laterally extending pins constitute one exemplary suspension means 104, that is to say, a means of suspending the catheter 20. Obviously those skilled in the art can envisage alternative suspension means 104, for example, a cradle instead of the arms.

In one embodiment, the separation and orientation step S2 may comprise suspending the catheters, by their funnels from a pair of infeed rails (not shown). The catheters 20 can then be transitioned from the infeed rails (not shown) to the processing rails 103 by being picked therefrom, by the gripping arms 101.

In another embodiment, not shown, the catheters 20 could hang directly from a pair of processing rails, provided the rails were suitably sized and spaced, with a distance between them that is smaller than the rim 24a, but larger than the catheter tube 20. As such, the suspension means, in the form of the gripping arms 101, is desirable, but not essential.

As shown in FIG. 1, at step S3, with the catheters remaining in their vertical orientation, hanging by the funnel 24 from the processing rails 103, a short handling sleeve 25 is formed around the catheter tube 23. Specifically, as shown in FIGS. 3 and 4, which depict alternative approaches to the formation of the handling sleeve 23, in a sheathing station, the sleeves 23 are formed around the funnel 24, the sleeves 23 extending proximally i.e. downward, because the catheters 20 are suspended by the funnel 24 so the proximal end 23 is beneath (substantially directly vertically beneath) the funnel 24.

In the embodiments shown, the funnel 24 is provided with a sleeve connection portion 24b, which, in this particular embodiment takes the form of a ribbed, generally cylindrical portion at the proximal end of the funnel (opposite the rib), where the funnel is connected to the catheter tube, having a diameter approximately equal to that of the rim 24a. Obviously its size and shape is purely exemplary.

The purpose of the sleeve connection portion 24b of the funnel 24 is to connect to the handling sleeve 25. The manner in which the sleeve may be formed around the sleeve connection is shown in FIGS. 3 and 4.

In a first embodiment of the method of step S3, with reference to FIG. 3, the catheter 20 is moved to a sheathing station along the rails (not shown), and two strips of film material 25a, 25b are advanced from rolls of the film material 105a, 105b. In this embodiment, the rolls are pre-formed with a point of weakness, in the form of a line of perforations 106 arranged at a short distance (e.g. 1 cm from the end which will become the top of the strip 25a, 25b). The strips 25a, 25b are then arranged opposing one another, sandwiching the catheter 20 therebetween. The strips 25a, 25b are elongate, and are attached to one another by welding along their opposed elongate edges.

In a second embodiment of the sheathing method of step S3, with reference to FIG. 4, the catheter 20 is again moved to a sheathing station along the rails (not shown), but in this embodiment just one strip of film material 25c is advanced from a roll of the film material 105c. The strip of film material 25c is wrapped around the catheter, with its top arranged so as to overly the sleeve connection portion 24b of the funnel 24 and the two elongate edges of the sleeve are welded together to form a tubular handling sleeve 25. In this embodiment too, the roll is pre-formed with a point of weakness, in the form of a line of perforations 106 arranged at a short distance (e.g. 1 cm from the end which will become the top of the strip 25a, 25b).

The perforations 106 define a sleeve attachment portion 26, which will remain attached to the funnel 24 in use, with the handling sleeve 25 consisting of the part beneath the point of weakness 106, which is releaseable from the funnel 24 by pulling it distally separating it from the sleeve attachment portion 26, which remains attached to the funnel.

Having formed the sleeve 25 around the catheter 20, whether in accordance with the method of the first or second embodiment, or indeed in by an alternative approach, in step S4, the sleeve is attached (in this example by tack welding the attachment portion 26) to the funnel 24. An embodiment of this approach is shown in FIG. 5, which shows the catheters 20 still hanging from their funnels 24, each held by suspension means 104, being advanced to a tack welding station. In the embodiment shown, the sleeves 25 are at this time held in position by grippers 107, which advance with the catheters 20. However, in practice, the sleeve could be formed simultaneously with the attachment step, S4.

In the sleeve attachment step, the sleeve attachment portion 26 is tack welded to the sleeve connection portion 24b of the funnel 24. To achieve this, in this particular embodiment, two spot welders 108 approach the catheter assembly 20 as it is advanced along the rails 103, suspended by the suspension means 104. The spot welders 108 approach the assembly from opposite sides and weld the sleeve attachment portion 26 to the sleeve connection portion 24b of the funnel 24, by forming spot welds 27 on opposing sides. Of course, only one side needs to be tack welded, but two welds 27 adds symmetry and redundancy.

With the sleeve attachment portion 26 welded to the funnel 24, but the sleeve 25 itself, readily removable from the sleeve attachment portion 26 (by virtue of the perforations 106), the sleeve 25 is releaseably attached to the funnel and the catheter 20 can continue to be processed in a vertical orientation, with the catheter tube 23 hanging down from the funnel 24 and the sleeve 25, too hanging off the funnel 24, making processing straightforward.

In one embodiment (not shown), a pouch could be formed around the catheter assembly 20 in its straight configuration; alternatively it could be picked, e.g. manually from the conveyer line and packed easily, as the sleeve remains attached.

However in this embodiment, at step S5, the orientation of the catheter 20 is changed from horizontal to vertical. To achieve this, the proximal ends 21 of the catheters 20 are lifted and the suspension means 104 is rotated such that the arms 101 are arranged horizontally rather than vertically.

At step S6, the catheter assemblies 20 are coiled (e.g. by a robot). The coiled catheters are arranged on carriers 50 in the form of H-shaped trays, with upstanding sides to constrain the coiled catheters 20, but open ends, to allow the catheter assemblies to slide off the trays. A fluid reservoir 10 in the form of a sachet of water is arranged on top of the coiled catheters in on the trays 50, and then, in step S7, as shown in FIG. 7, the coiled catheters and sachets of water are introduced into the open base of a pouch 30. As shown in S8, this is completion of the sleeve conversion and catheter infeed process.

In this embodiment, the catheter tube 23 is a hydrophilic thermoplastic elastomer (TPE), and the handling sleeve 25 and handling sleeve elements 25a, 25b are formed from a film of low-density polyethylene (LDPE). The funnel 24 is made from polyvinyl chloride (PVC).

In this embodiment, the pouch 30 comprises a front opaque wall 31a, a rear opaque wall 31b, and a peripheral seal joining the periphery of the walls 31a, 31b together to form the pouch. The peripheral seal comprises a base 32, which is sealed only after completion of the sleeve conversion and catheter infeed process S8, along with a right lateral edge 33, a left lateral edge 34, and an upper edge 35, each of which is sealed before the catheter is introduced into the pouch. The left lateral edge 34 and right lateral edge 33 are defined as the left and right sides of the pouch when viewing the pouch 30 with the rear wall 31b behind the front wall 31a, the base 32 at the bottom of the pouch 30 and the upper edge 35 at the top of the pouch 30. The peripheral seal thus defines a pouch 30 that is generally rectangular and suitably has a width between the lateral edges of between 60-110 mm, for example about 100 mm, e.g. 95 mm, and a height from the base 32 to the upper edge 35 of between 100 to 250 mm, for example about 200 mm, e.g. 197 mm. The base 32 defines the bottom of the pouch in use, and the upper edge 35 the top. However, in other embodiments alternative shapes and sizes could be conceived, such as an elliptical or circular pouch.

In this particular embodiment, after sealing the base 32 of the pouch, or concurrently, a join 36 is formed between the two walls 31a, 31b. The join 36 is a circular weld provided at point equidistant between the lateral edges 33, 34 of the peripherals seal and at a distance above the base 32 extending from 45 to 55 mm. The join 36 may have a radius between 1% and 10% the height of the pouch, for example 5%. In other embodiments the join may be provided in different shapes and sizes, at different locations, or could potentially even be integrated into a continuous seal with the peripheral seal.

The pouch also comprises a window 37 in one of the walls, which allows a user to see inside the pouch. In this particular embodiment, the window 37 is transparent elongate and generally rectangular with a length between 20-50% the height of the pouch 30, for example 33%, and width between 5-25% the width of the pouch 30, for example 12.5%. In this example, the window 37 has semi-circular ends, but in other embodiments they may be any suitable shape, such as flat ends or irregularly shaped ends. The window 37 is arranged parallel to the right lateral edge 33, and a gap is provided between the edge of the window 37 and right lateral edge 33 equivalent to between 5-20% the width of the pouch, for example 10%. The bottom of the window 37 is separated from the base of the peripheral seal 32 by a distance equal to between 5-20% the height of the pouch, for example 10%. In other embodiments, translucent windows may be used and additionally alternative shapes, sizes and positions or angles of the window are contemplated (for example say at an angle of up to 20 degrees with respect to the edges).

In this particular embodiment, each wall 31a, 31b comprises a plurality of layers of foil; in one specific embodiment a 97 micron 2-layer foil of 12 micron PET and 60 micron LLDPE. In other embodiments, the walls 31a, 31b may only comprise a single layer, and different plastics materials may be used interchangeably.

As mentioned above, the walls 31a, 31b are opaque. This may be achieved by forming each wall 31a, 31b from one or more opaque layers of material or printing an opaque pattern onto one of the layers. The window 37 in the front wall 31a, may be formed by laminating an opaque layer of material onto a transparent wall, wherein the opaque layer has an aperture corresponding to the window 37. Alternatively, a transparent wall may be printed on to block light from passing through the wall, rendering it opaque in all places except for the location of the window 37. Further alternatives may also be contemplated and achieved, for example, providing an aperture corresponding to the window 37 in an opaque wall, and bonding a transparent layer so as to cover the aperture and achieve a continuous front wall 31a with a window 37, or starting with a multi-layer foil with an opaque layer and a transparent layer and removing part of the opaque layer of foil to form the transparent window.

In this particular embodiment, the pouch also comprises an interaction region. The interaction region forms the top part of the pouch 30 and spans between the right lateral edge 33 and left lateral edge 34. The interaction region is used to provide access to the pouch through/near to the upper edge 35. In this embodiment, the interaction region comprises a tapered tear away region 38 and a sealing arrangement 39. The tear-away region 38 comprises a tear start 40 on the left lateral edge, about 2 cm below at the point the upper edge 35 and left lateral edge 34 meet, and a tear stop 41 at a point in the pouch 30 about 1 cm below where the upper 35 and right lateral 33 edges meet. The distance from the tear stop 41 and the right lateral edge 33 being between 1-10% the width of the pouch, for example 5%. The tear start 40 comprises a notch to provide an area of weakness in the walls 31a, 31b allowing the tear away region 38 to be separated from the pouch 30 by tearing the walls apart from the tear start 40 to the tear stop 41. Thus the tear is provided beneath the location of the upper edge 35 and thus the pouch 30 is opened provided access to its contents. The tear stop 41 comprises a small aperture in the walls 31a, 31b to prevent further tearing of the pouch 30 and separation of the tear away region 38 from the pouch 30. In order to provide the tapered shape, the tear away region 38 extends upwards on the left side approximately 50% more than it does on the right side. The left side of the tear away region 38 comprises a circular aperture 42 sized to allow a finger to pass through and grip the tear away region 38. To ensure the pouch is fully sealed, the front wall 31a and rear wall 31b are sealed to one another around the periphery of the tear stop 41 and circular aperture 42. In other embodiments, other seals such as a zip-lock arrangement may be used to provide access to the pouch, and of course, the sizes and shapes set out above are exemplary.

In this embodiment, the sealing arrangement 39 extends between the left lateral edge 34 and right lateral edge 33 of the peripheral seal just below the tear away region 38. The sealing arrangement 39 comprises first and second sealing members (not shown) provided on an interior surface of the walls 31a, 31b of the pouch 30. In this embodiment, the sealing members comprise corresponding sealing grooves and ridges to allow the sealing members to be press-fit together to form a water-tight seal and pulled apart again if necessary. In other embodiments, a single adhesive sealing member may be used, or a sealing member may be provided on the outside of the pouch to allow the pouch to be folded or rolled and sealed accordingly and of course, alternative sealing mechanisms like a hook-and-loop or hook-and-hook seal or zip-lock could be used.

In this embodiment, the fluid reservoir 10 is an elongate rectangular burstable sachet of water, but in other embodiments any suitable reservoir may be used. The fluid reservoir 10 has a height equal to between 60-90% the height of the pouch 30, for example 80%, and a width between 5-25% the width of the pouch 30, for example 15%. In this embodiment, the fluid contained within the reservoir 10 is water. In other embodiments, many other types of reservoir would be suitable including different shapes and sizes of reservoir, or non-burstable reservoirs and other external sources of fluid. In addition, other fluids could be used, for example lubricants, gels, oils or the like.

In this embodiment, the catheter 20 has a length of between 30-50 cm, for example 40 cm, and the handling sleeve 25 is between 10 and 30% the length of the catheter 20, for example 20-25%, e.g. 11 cm.

In this embodiment, the perforations 106 extend perpendicularly to the length of the catheter 20 across the width of the sleeve 25 to separate it from the sleeve attachment portion 26. In this embodiment, the perforations 26 comprise a row of elongate holes extending around the circumference of the handling sleeve 25. The holes have a width and length perpendicular to the width and length of the handling sleeve 25 respectively. In this embodiment, the holes are slits and as such they are one dimensional and have an aspect ratio (length:width) that is much greater than 2:1 and effectively 1:0.

In this embodiment, the holes are separated by a distance that is no more than the length of each hole, and in this example the holes are separated by 1 mm. In this embodiment, the length of each hole is 2 mm. As such, the perforations 106 provide for a point of weakness in the handling sleeve 25 that is sufficient to allow easy separation of the handling sleeve 25 from the funnel 24.

In this embodiment, the catheter 20 is provided with a hydrophilic coating, this is activated when it contacts the fluid held in the reservoir 10. In other embodiments, any suitable catheter length or type may be used. The inner surface of the handling sleeve 25 may be hydrophobic and may comprise an embossed pattern. This reduces friction between the catheter 20 and the handling sleeve 25 allowing easier arrangement of the sleeve along the catheter 20 during use. In one example, the embossed pattern is a linen pattern.

The catheter 20 is arranged within the pouch 30 in a curved and coiled configuration, with the distal end 22 adjacent to the inner left lateral edge 33 near the upper 35 edge of the pouch 30, just beneath the sealing arrangement 39. The catheter 20 extends down the inside of the pouch 30 into a broadly elliptical anti-clockwise coil (as viewed with left lateral edge 34 on the left, and upper edge 35 at the top). The coil having a single turn around the inner perimeter of the left lateral edge 34, base 23, and right lateral edge 33 of the pouch, with the proximal end 21 of the catheter 20 resting on the inside of the catheter's coil, adjacent to the left lateral edge 34 and base 32 of the pouch. Notably, the majority of the handling sleeve 25 is arranged above the join 36, and as such, the perforations 26 are arranged above the join 36. As the catheter 20 curves and extends from left to right, the catheter tube 23 runs underneath the join, in a channel defined by the join and the internal base of the peripheral seal, then runs upwards to the side of the join, before returning over the top of the join and finally extending downwardly such that the proximal end 21 is lower than and on the left side of the join 36. In short, the catheter 20 is coiled (or curled) around the join 36 (albeit not tightly coiled). In other embodiments, the position of the catheter 20 may be different, in particular, the catheter 20 may not coil or curl but may simply curve within the pouch 30. On the other hand, embodiments of the invention may have a catheter 20 that has multiple turns of coil.

In this embodiment, the fluid reservoir 10 is also placed within the pouch 30 at a position corresponding to the window 37. The reservoir 10 is placed over the catheter 20 so that the reservoir 10 is visible through the window 37 and the reservoir 10 obstructs any view of the catheter 20 through the window 37. In other embodiments, the reservoir 10 may not need to be placed over the catheter 20 as the position of the catheter 20 in the pouch 30 may not correspond to the window 37.

Because the catheter 20 is coiled around the inner perimeter of the pouch 30, the join 36 is located within the coil of the catheter 20. Therefore, part of the catheter tube 23 passes between the join 36 and the base 32 of the pouch. In other embodiments, the join 36 may be provided before the peripheral seal, or at any point of the assembly process.

As will be appreciated, the above is an example and in other embodiments the pouch 30 may be formed in different ways with the steps performed in different orders. For example, while the peripheral seal and join 36 have been described as formed by a weld, any suitable means of joining the walls could be used, for example, an adhesive may be used.

Referring to FIGS. 9 to 11, this first embodiment of a packaged catheter assembly 100 may be unpackaged as described below.

First, the fluid reservoir 10 is located within the pouch 30 with the assistance of the window 37, though which the reservoir 10 can be seen. Fluid is then released from the reservoir 10 into the pouch 30. In this embodiment, the reservoir 10 is a burstable sachet and fluid may be released from it by applying manual pressure to the reservoir 10. The window 37 provides a visual reference to show when the reservoir has been successfully burst and fluid released into the pouch 30. In particular, it can be seen that sufficient fluid has been released to fill the channel defined by the weld 36 and the internal base 32 of the peripheral seal. In other embodiments, the fluid may be released from other means.

Referring to FIGS. 9 and 10, the fluid released from the reservoir 10 collects in a pool at the bottom of the pouch 30, as shown by the hashing. In this embodiment, the fluid fills the pouch 30 up to a fill level corresponding to the join 36. As such, in this embodiment, the join 36 and base 32 define a channel that is completely full of fluid. However, in other embodiments, the fluid may only partially fill the channel, for example filling the channel between 60-95%, or may fill the pouch above the level of the join 36.

The released fluid is also visible through the window 37, as the base of the window 37 corresponds to a location on the pouch 30 that is slightly lower than the join 36. In other embodiments, the window may comprise an indicator region that shows the presence of fluid in the pouch. For example, a fluid-sensitive colour changing strip such that direct visualisation of the fluid is not required. A fill-level marker could also be provided to show that sufficient fluid has been released.

In this embodiment, the contents of the pouch 30 are then accessed by tearing the top of the pouch 30 using the tear away region 38. The aperture 42 is grasped in one hand, and the rest of the pouch 30 in another, and the tear away region 38 is torn from the tear start 40 to the tear stop 41. Thus, the pouch is torn between the left lateral edge 34 and right lateral edge 33 at a location below the upper edge 35, as such, an opening is formed in the pouch 30 that may be used to access its contents. Advantageously, the tear away region 38 is not fully separated from the pouch 30 which reduces the number of separate parts and makes the pouch 30 easier to handle. Other embodiments may feature other ways to access the pouch for example a zip-lock seal.

Referring to FIG. 10, the catheter 20 may then be withdrawn from the pouch 30. As described above, in this embodiment the catheter 20 is coiled in the pouch 30, and the distal end 22 is provided adjacent the upper edge 35 of the pouch 30. As such, the distal end 22 is above the fill line of the fluid released into the pouch 30 and is not wetted by the fluid. The distal end 22 therefore provides a dry and easy to access point which may be used to withdraw the catheter 20 from the pouch 30. In this embodiment, the majority of the handling sleeve 25, i.e. between 60-100%, for example 70% of the sleeve 25 is also above the fill line and is not wetted. Thus, the sleeve 25 also provides a convenient dry surface to handle the catheter 20. In particular, the distal handling sleeve element 25b is entirely above the fill line and is therefore dry.

In this embodiment, the distal end 22 is pulled upwards through the opening in the upper edge 35 of the pouch 30. As the distal end 22 is pulled out of the pouch 30, the catheter tube 23 is uncoiled and also pulled upwards towards the upper edge 35 of the pouch 30. However, as the join 36 is arranged within the coil of the catheter 20, the tube 23 is prevented from moving directly upwards by the join 36. The join 36 therefore causes the tube 23 to be withdrawn from the pouch 30 along a path that includes passing through the channel defined by the join 36 and base 32. As such, the entire remainder of the tube 23 to the proximal tip 21 passes though the pool of fluid in the channel and the hydrophilic coating of the catheter 20 is activated by the fluid. Furthermore, the proximal end 21 of the catheter 20 is the final part of the catheter 20 to pass through the channel and be withdrawn from the pouch 30. Thus, the catheter 20 is activated by the fluid as it is withdrawn and the withdrawal is convenient and easy as only a dry part of the catheter 20 needs to be touched during withdrawal. As the catheter 20 passes through a pool of fluid it is more completely wetted than would be the case for a fluid held within another medium such as a foam. In other embodiments, the catheter 20 need not be withdrawn directly upwards but may be pulled out to sideways from the pouch, for example from an opening provided at the side of the pouch, but, of course, above the weld, so as to reduce the prospect of spillage. Further the catheter 20 may not directly touch the join 36 and may instead be prevented from moving upwards by the convergence of the walls 31a, 31b as they approach the join 36.

Once the catheter is fully withdrawn, as shown in FIGS. 11 and 12, the catheter may be used by inserting the proximal end 21 into the urethra until urine begins to flow through the catheter 20. The (largely dry) handling sleeve 25 is released from the funnel 24, as shown in FIG. 12 by simply pulling it towards the proximal end 21 of the catheter 20, such that it breaks away from the sleeve attachment portion 26 at the point of weakness 106.

The handling sleeve 25 can then be used to assist in the insertion of the catheter 20. The urine may then be directed out of the catheter 20 by the funnel 24.

The catheter 20 may be placed back inside the pouch 30 in a coiled configuration after use. The handling sleeve 25 and funnel 24 provide for dry surfaces that can be used to re-coil the catheter 20 and place it into the pouch 30.

The sealing arrangement 39 can then be used to reseal the pouch 30 between the left lateral edge 34 and right lateral edge 33 by pressing together the two corresponding sealing elements (not shown). This provides for a more convenient disposal of the catheter 20 after use and ensures the fluid released into the pouch 30 does not subsequently leak out of the pouch 30. In other embodiments, alternative sealing arrangements may be used, such as an external flap with adhesive that allows the top of the pouch 30 to be rolled or folded and sealed. Alternatively, no sealing arrangement may be used where the pouch 30 is intended to be disposed of immediately after use.

Referring to FIG. 13, a third embodiment of a packaged catheter assembly 100 is shown. The third embodiment shares many of the features of the first and second embodiment described above, and so only differences in the features are described below, and like numerals are used to denote like features.

In this third embodiment, the film material used to form the sleeve 25 is not provided with a line of weakness. Instead, when the welding apparatus 108 forms the spot weld 27, this creates a weakness between the welded portion 27 and the remainder of the sleeve. As such, simply pulling the sleeve 25 detaches it from the welded portion 27.

Of course, the method of use of the catheter assembly 10 of the third embodiment is identical to that of the first and second embodiment, and the sleeve 25 could be formed by the method of either the first or second embodiment, or indeed formed separately and threaded onto the catheter 20, from the proximal end 21, up to the funnel 24, as shown in FIG. 14.

Referring to FIG. 15, a fourth embodiment of a catheter assembly is shown. The fourth embodiment shares many of the features of the first to third embodiments described above, and so only differences in the features are described below, and like numerals are used to denote like features.

In this fourth embodiment, as with the third embodiment, the film material used to form the sleeve 25 is not provided with a line of weakness. Instead, rather than welding a sleeve attachment portion to the funnel, in this embodiment, a spot of adhesive 28 is provided on the funnel 24 and the sleeve 25 adheres, releaseably to the funnel, via the adhesive. Of course, those skilled in that art, can easily, e.g. by trial and error, select a suitable adhesive, such as an epoxy resin or silicone adhesive that will provide sufficient tackiness to hold the sleeve 25 in place, but release it when pulled by a user. A similar result could be achieved by tack-welding the sleeve 25 to the funnel 24, such that the bond between the weld (which would be at the point indicated by the adhesive 28) and the remainder of the sleeve 25 is stronger than that between the weld and the funnel, allowing the entire sleeve to come away when pulled.

Of course, the method of use of the catheter assembly 10 of the fourth embodiment is identical to that of the embodiments described above, and the sleeve 25 could be formed by the method of either the first or second embodiment, or indeed formed separately and threaded onto the catheter 20, from the proximal end 21, up to the funnel 24, as shown in FIG. 14.

Referring to FIG. 16, a fifth embodiment of a catheter assembly is shown. The fifth embodiment shares many of the features of the embodiments described above, and so only differences in the features are described below, and like numerals are used to denote like features.

In this fifth embodiment the film material used to form the sleeve 25 is not provided with a line of weakness, rather, in order that it is releasably attached to the funnel, it is formed so as to have an inner diameter which is a tight-fit around the sleeve connection portion 24b of the funnel. In this embodiment, the sleeve connection portion 24b is formed from a grippy and resilient material, such as rubber or silicone-rubber which could be contracted by the sleeve 25. Alternatively, a similar effect could be achieved by providing the sleeve connection portion 24b from a material that that statically attracts the sleeve 25.

Of course, the method of use of the catheter assembly 10 of the fifth embodiment is identical to that of the embodiments described above, and the sleeve 25 could be formed by the method of either the first or second embodiment, or indeed formed separately and threaded onto the catheter 20, from the proximal end 21, up to the funnel 24, as shown in FIG. 14.

Referring to FIG. 17, a sixth embodiment of a catheter assembly is shown. The sixth embodiment shares many of the features of the embodiments described above, and so only differences in the features are described below, and like numerals are used to denote like features.

In this sixth embodiment the film material used to form the sleeve 25 is not provided with a line of weakness, nor does it have an inner diameter that is a tight fit around the sleeve connection portion 24b of the funnel. Instead, after arranging the sleeve 25 around the funnel 24, two spot welds 28a are made attaching one side of the sleeve to the other side, and thereby creating a region with a smaller inner diameter, between the welds. These welds are arranged just above the sleeve connection portion 24a of the funnel 24, to releasably attach the sleeve 25 to the funnel.

Of course, the method of use of the catheter assembly 10 of the sixth embodiment is identical to that of the embodiments described above, and the sleeve 25 could be formed by the method of either the first or second embodiment, or indeed formed separately and threaded onto the catheter 20, from the proximal end 21, up to the funnel 24, as shown in FIG. 14. Preferably the sleeve is made in accordance with the method of the first or second embodiment, and the spot welds 28a between opposing sides of the sleeve 25 are made at the same time as the sleeve 25 is formed. Obviously the provision of two spot welds between the sides of the sleeve 25 is not essential, and one spot weld would suffice.

Referring to FIGS. 18 and 18A, a seventh embodiment of a catheter assembly is shown. The seventh embodiment shares many of the features of the embodiments described above, and so only differences in the features are described below, and like numerals are used to denote like features.

In this seventh embodiment the film material used to form the sleeve 25 is not provided with a line of weakness, nor does it have an inner diameter that is a tight fit around the sleeve connection portion 24b of the funnel. Instead, after arranging the sleeve 25 around the funnel 24, the uppermost region of the sleeve is heat-shrunk around the sleeve connection portion 24b of the funnel creating a region with a smaller inner diameter, which is releaseably attached to the funnel 24, as it remains in connection under normal conditions (on account of the tight fit owing to the heat-shrinking reducing the inner diameter), but when pulled axially towards the proximal end 21 of the catheter 20 will release (optionally splitting at its uppermost end, when pulled).

Of course, the method of use of the catheter assembly 10 of the seventh embodiment is identical to that of the embodiments described above, and the sleeve 25 could be formed by the method of either the first or second embodiment, or indeed formed separately and threaded onto the catheter 20, from the proximal end 21, up to the funnel 24, as shown in FIG. 14.

Referring to FIG. 19, an eighth embodiment of a catheter assembly is shown. The eighth embodiment shares many of the features of the embodiments described above, and so only differences in the features are described below, and like numerals are used to denote like features.

In this eighth embodiment the film material used to form the sleeve 25 has a line of weakness 106, and, like in the first and second embodiments, it is welded to the funnel 24. The difference in this embodiment is that, as shown in the drawing, rather than a separate spot-weld, the weld that attaches the sleeve 25 to the funnel 24 is a peripheral weld, that also joins the elongate strips together along the top.

Of course, the method of use of the catheter assembly 10 of the eighth embodiment is identical to that of the embodiments described above, and the sleeve 25 could be formed by the method of either the first or second embodiment, or indeed formed separately and threaded onto the catheter 20, from the proximal end 21, up to the funnel 24, as shown in FIG. 14, before being welded along its peripheral top edge to the funnel 24.

Referring to FIG. 20, a ninth embodiment of a catheter assembly is shown. The ninth embodiment shares many of the features of the embodiments described above, and so only differences in the features are described below, and like numerals are used to denote like features.

In this ninth embodiment the film material used to form the sleeve 25 has no line of weakness and to releasably connect it to the funnel 24, a separate part-circular clip 60 is provided, which is a tight fit around the funnel 24, and is clipped around the funnel, after the sleeve 25 has been arranged around the funnel 24, so as to sandwich the sleeve 25 between the clip and the funnel 24.

Of course, the method of use of the catheter assembly 10 of the ninth embodiment is identical to that of the embodiments described above, and the sleeve 25 could be formed by the method of either the first or second embodiment, or indeed formed separately and threaded onto the catheter 20, from the proximal end 21, up to the funnel 24, as shown in FIG. 14, before being clipped in place.

Referring to FIGS. 21 and 21A, a tenth embodiment of a catheter assembly is shown. The tenth embodiment is most similar to the ninth embodiment described above, and so only differences in the features are described below, and like numerals are used to denote like features.

As in the ninth embodiment the film material used to form the sleeve 25 has no line of weakness and to releasably connect it to the funnel 24, and similarly, in this tenth embodiment, a clip 61 is provided. The difference is that in this embodiment, the funnel 24 is adapted, such that two diametrically opposed clips 61 are integrally formed on its outside surface, each clip 61 being of the shirt-clip type provided on pens, to clip them to a shirt pocket. As with the separate clip of the ninth embodiment, the clips 61 of the tenth embodiment are springy and biased towards a position in which they sandwich the sleeve 25 between the clip and the funnel 24 (such that the sleeve can be released). Incidentally, the clips 60, 61 of the ninth and tenth embodiments could allow the sleeves to be reattached after use, if that were desired.

Of course, the method of use of the catheter assembly 10 of the tenth embodiment is identical to that of the embodiments described above, and the sleeve 25 could be formed by the method of either the first or second embodiment, or indeed formed separately and threaded onto the catheter 20, from the proximal end 21, up to the funnel 24, as shown in FIG. 14, before being clipped in place.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

The one or more embodiments are described aboe by way of example only. Many variations are possible without departing from the scope of protection afforded by the appended claims.

1. A method of processing a catheter assembly comprising a catheter tube coupled to a funnel and a handling sleeve releasably attached to the funnel; the method comprising engaging the funnel, such that the catheter tube hangs down from the funnel.

2. A method according to claim 1 comprising engaging the funnel, such that the catheter tube hangs down from the funnel and releasably attaching the handling sleeve thereto.

3. A method according to claim 1 or 2 wherein the handling sleeve is attached via a sleeve-attachment portion.

4. A method according to any preceding claim wherein the handling sleeve is formed of a flexible film.

5. A method according to claim 3 wherein the handling sleeve and the sleeve attachment portion are formed of a flexible film and wherein a point of weakness is provided between the handling sleeve and the sleeve-attachment portion.

6. A method according to any preceding claim wherein the handling sleeve is a short sleeve which extends extend along less than 50% of the distance to the tip of the catheter tube, whereby a distance of at least 50% from the tip of the catheter towards the funnel is exposed.

7. A method according to any preceding claim comprising engaging the funnel with processing rails.

8. A method according to claim 7 comprising suspending the funnel from suspension means, wherein the suspension means are engaged with the processing rails.

9. A method according to any preceding claim comprising separating and orienting a plurality of catheters in bulk, then picking a catheter, the method comprising orienting the catheter assemblies vertically.

10. A method according to any preceding claim comprising advancing a catheter assembly along rails to a sheathing station and attaching the handling sleeve to the funnel in the sheathing station.

11. A method according to claim 10 comprising wrapping a sheet of film material around the catheter tube and joining opposing elongate edges of the sheet to form the handling sleeve, or sandwiching the catheter tube between two sheets of film material and joining the two sheets along two elongate edges of each sheet to form the handling sleeve.

12. A method according to claim 11 wherein the or each sheet of film material is provided with a point of weakness extending perpendicular to their elongate edges, such that formation of the handling sleeve can simultaneously form a sleeve attachment portion, separated from the handling sleeve by the point of weakness.

13. A method according to claim 5 or any of claims 6 to 12 when dependent on claim 5, comprising permanently attaching the sleeve-attachment portion to the funnel by welding or gluing, wherein the handling sleeve is releaseably attached to the sleeve attachment portion that is permanently attached to the funnel.

14. A method according to claim 5 or any of claims 6 to 12 when dependent on claim 5 wherein the handling sleeve of the sleeve is releaseably attached to the sleeve-attachment portion that is permanently attached to the funnel by means of a frangible connection, the frangible connection formed by welding the sleeve to the funnel, such that the region where the sleeve is welded to the funnel remains permanently attached to the funnel forming a sleeve attachment portion, but the remainder of the sleeve breaks away from the welded region forming the handling sleeve.

15. A method according to claim 1, 2 or any one of claims 6 to 11 comprising releaseably attaching the entire handling sleeve to the funnel, such that the entire sleeve can be released from the funnel, without leaving any portion attached.

16. A method according to any preceding claim comprising reorienting the catheter, after releaseably attaching the handling sleeve from a vertical configuration to a horizontal configuration.

17. A method according to claim 16 comprising coiling the catheter, with the catheter in the horizontal configuration, and arranging the coiled catheter on a carrier.

18. A method according to claim 17 comprising introducing the coiled catheter and a fluid reservoir into a pouch on a carrier; wherein the coiled catheter is introduced through a side or end that is opposite to a side intended to be opened, which is provided with an opening mechanism and resealing means.

19. A method according to claim 18 comprising sealing the pouch after introducing the coiled catheter and the fluid reservoir; and forming a join between the two walls of the pouch, such that the join and an internal base of the peripheral seal may define a channel to be filled with fluid prior to withdrawal of the catheter from the pouch, in use.

20. A catheter assembly comprising a catheter tube coupled to a funnel, and a handling sleeve; wherein the handling sleeve is formed from a flexible film material and is releasably attached to the funnel.

21. A catheter assembly according to claim 20 wherein the handling sleeve is a short sleeve that, when attached, extends along less than 70% of the distance to the tip of the catheter tube, whereby a distance of at least 30% from the tip of the catheter towards the funnel is exposed.

22. A catheter according to claim 20 or 21 wherein the handling sleeve is irreversibly detachable from the funnel and wherein the handling sleeve is movable along the length of the catheter.

23. A catheter according to claim 20 or 21 wherein the handling sleeve is frangibly attached via a sleeve-attachment portion; the sleeve-attachment portion and the handling sleeve being separable by breaking a point of weakness between the handling sleeve and the sleeve-attachment portion; the point of weakness being provided by a plurality of perforations; the perforations comprising a row of holes extending around the circumference of the handling sleeve; and each hole forming the perforations being a slit.

24. A catheter assembly according to claim 23 wherein the separation force required to detach the handling sleeve from the sleeve attachment portion is less than the separation force required to detach the sleeve attachment portion from the funnel.

25. A catheter assembly according to any of claims 20 to 24 wherein the catheter assembly is provided in a pouch; the pouch being sealed and sterilized.

26. A catheter assembly according to any of claims 20 to 24 wherein the handling sleeve is made from any one or more of: polypropylene (PP), low density polyethylene (LDPE), high density polyethylene (HDPE) or biaxially orientated polypropylene (BOPP).
